(19) United States
(12) Patent Application Publication
(10) Pub. No.: US 2025/0019463 A1
(43) Pub. Date: Jan. 16, 2025

(54) METHOD FOR THE TREATMENT OF PROGRESSIVE CHRONIC INTERSTITIAL LUNG DISEASE

(71) Applicant: UCB Biopharma SRL, Brussels (BE)

(72) Inventors: Timothy Scott JOHNSON, Berkshire (GB); Ian Trollope JAMES, Berkshire (GB); Patricia J. SIME, Richmond, VA (US); Thomas Henry THATCHER, Richmond, VA (US)

(21) Appl. No.: 18/711,286

(22) PCT Filed: Nov. 17, 2022
(86) PCT No.: PCT/EP2022/082299

§ 371 Date: May 17, 2024

Related U.S. Application Data
(60) Provisional application No. 63/264,276, filed on Nov. 18, 2021.

(51) Int. Cl. C07K 16/40; A61K 39/00; A61P 11/00

(52) U.S. Cl. CPC C07K 16/40; A61P 11/00; A61K 2039/505; C07K 2317/76

(57) ABSTRACT

The present invention relates to an anti-transglutaminase type 2 antibody that blocks transamidase activity of the enzyme for use in the treatment of progressive chronic interstitial lung disease, such as Idiopathic pulmonary fibrosis (IPF).

### FIELD OF INVENTION

The present invention relates to an anti-transglutaminase type 2 antibody that blocks transamidase activity of the enzyme for use in the treatment of progressive chronic interstitial lung disease, such as Idiopathic pulmonary fibrosis (IPF).

### BACKGROUND OF THE INVENTION

Tissue transglutaminase or transglutaminase type 2 (TG2) is an enzyme which forms crosslinks between proteins via epsilon (gamma-glutamyl) lysine di-peptide bonds. Elevated expression of TG2 leads to aberrant protein cross-linking which has been associated with several pathologies including various types of tissue scarring and fibrosis, the formation of neurofibrillary tangles in several brain disorders and resistance to chemotherapy in some cancers. Various TG2 inhibitors, such as small molecules, silencing RNA or antibodies (e.g. Siegel et al., 2007, Wang et al., 2020, WO2006100679, WO2012146901 or WO2013175229), have been disclosed for the possible treatment of TG2-mediated disorders.

Progressive chronic interstitial lung diseases include Idiopathic pulmonary fibrosis (IPF) which is characterized by the deposition of scar tissue in the lung interstitium resulting in alveolar membrane thickening, progressive decline in lung function, and eventually death. The overall prognosis after diagnosis with IPF is typically poor as the disease progresses steadily, ultimately resulting in death.

Increased levels of TG2 and LOXL2 have been associated with progressive chronic interstitial lung diseases, such as Idiopathic pulmonary fibrosis (Olsen et al., 2011, Olsen et al., 2020, Philp et al., 2018). The overexpression or overactivity of TG2 in IPF-derived fibroblasts results in an increase in epsilon (gamma-glutamyl) lysine crosslinks between proteins of the extracellular matrix (ECM). TG2 and LOXL2 manipulation via use of small inhibitors or use of null mice during the induction of fibrosis reduces lung inflammation and fibrosis in bleomycin-treated animals (see Olsen et al., 2011, Olsen et al., 2014, Raghu 2017, Vaidya et al., 2017, Philp et al., 2018). For instance, Fibroblast adhesion and proliferation assays have shown that coincubation of TG2 with cystamine (a pan TG inhibitor) abrogated the increased adhesion linked to TG2 (see Philp 2018). While the primary roles of TG2 crosslinking activity in fibrotic remodelling appear to be via slowing turnover of extracellular matrix (ECM) by the incorporation of protease resistant intramolecular crosslinks in ECM proteins and the local activation of TGFβ1, there is also some emerging evidence that in lung cells the previously ascribed none enzymatic role of TG2 in cell adhesion may be modulated by blocking transamidation activity as exemplified by the use of cystamine to reduce lung fibroblast adhesion in vitro.

Two approved therapies, pirfenidone (which appears to partially function via modulation of TGFβ1 related pathways including collagen synthesis) and nintedanib (targeting multiple tyrosine kinases), are effective in slowing the progression of disease in some patients, and additional therapeutic options are urgently needed (Margaritopoulos et al., 2016). However, there remains a need to identify further effective therapies for use in treatment and prevention of progressive chronic interstitial lung disease, such as IPF as well as for use in the treatment of a subject having a lung fibrosis associated with COVID infection or in the prevention of the development of a lung fibrosis in a patient suffering from COVID infection.

### SUMMARY OF THE INVENTION

It is an object of the present invention to provide a specific anti-transglutaminase 2 (anti-TG2) antibody for use in the treatment of progressive chronic interstitial lung disease, such as Idiopathic Pulmonary Fibrosis (IPF) or for use in the prevention of the development of progressive chronic interstitial lung disease, such as Idiopathic Pulmonary Fibrosis (IPF). Alternatively, the present invention provides a specific anti-transglutaminase 2 (anti-TG2) antibody for use in the treatment of a subject having a lung fibrosis associated with COVID infection or in the prevention of the development of a lung fibrosis in a patient suffering from COVID infection.

In a second aspect, the invention provides a method for treating or for preventing the development of progressive chronic interstitial lung disease comprising administering a therapeutically effective amount of an anti-TG2 antibody. Alternatively, the present invention provides a method for treating a subject having a lung fibrosis associated with COVID infection or for preventing the development a lung fibrosis in a patient suffering from COVID infection, comprising administering a therapeutically effective amount of an anti-TG2 antibody.

In a third aspect, the invention relates to the use of an anti-TG2 antibody for the manufacturing of a medicament for the treatment of a progressive chronic interstitial lung disease or for the prevention of the development of progressive chronic interstitial lung disease, such as Idiopathic Pulmonary Fibrosis (IPF). Alternatively, the present invention relates to the use of an anti-TG2 antibody for the manufacturing of a medicament in the treatment of a subject having a lung fibrosis associated with COVID infection or the prevention of the development a lung fibrosis in a patient suffering from COVID infection.

### Definitions

The entire document is intended to be related as a unified disclosure, and it should be understood that all combinations of features described herein are contemplated, even if the combination of features are not found together in the same sentence, or paragraph, or section of this document. With respect to aspects of the invention described or claimed with “a” or “an”, it should be understood that these terms mean “one or more” unless context unambiguously requires a more restricted meaning. The term “or” should be understood to encompass items in the alternative or together, unless context unambiguously requires otherwise. If aspects of the invention are described as “comprising” a feature, embodiments also are contemplated “consisting of” or “consisting essentially of” the feature.

The term “Tissue transglutaminase”, “Transglutaminase type 2” or “TG2” refers to an enzyme which forms crosslinks between proteins via epsilon (gamma-glutamyl) lysine di-peptide bonds. TG2 refers to a protein that typically has the amino acid sequence as set out in the UniProt entry P21980 (SEQ ID NO: 41), i.e. human TG2. The term “TG2” may also refer to protein which is (a) a derivative having one or more amino acid substitutions, modifications, deletions or insertions relative to the amino acid sequence of SEQ ID NO: 41 which retains the activity of TG2, or (b) a variant thereof, such variants typically retain at least about 75%, 80%, 85%, 90%, 91%, 92%, 93%, 94% or 95% identity to SEQ ID NO: 41 (or even about 96%, 97%, 98% or 99% identity to SEQ ID NO: 41). The protein TG2 is encoded by the gene Tgm2.

The term “anti-TG2 antibody”, as used herein, is intended to be an antibody molecule which binds TG2 and block its transamidase activity to prevent crosslinking. Examples of such antibodies are described in WO2013175229. Without any limitation, an anti-TG2 antibody that can be used according to the present invention comprises for instance a light chain variable region as defined in SEQ ID NO: 24 and a heavy chain variable region as defined in SEQ ID NO: 37.

The term “antibody” as used herein includes, but is not limited to, monoclonal antibodies, polyclonal antibodies and recombinant antibodies that are generated by recombinant technologies as known in the art. “Antibody” include antibodies of any species; such as human antibodies of any isotype, including IgG1, IgG2a, IgG2b, IgG3, IgG4, IgE, IgD and antibodies that are produced as dimers of this basic structure including IgGA1, IgGA2, or pentamers such as IgM and modified variants thereof; non-human primate antibodies, e.g. from chimpanzee, baboon, rhesus or cynomolgus monkey; rodent antibodies, e.g. from mouse, or rat; rabbit, goat or horse antibodies; camelid antibodies (e.g. from camels or llamas such as Nanobodies™) and derivatives thereof; antibodies of bird species such as chicken antibodies; or antibodies of fish species such as shark antibodies. The term “antibody” also refers to “chimeric” antibodies in which a first portion of at least one heavy and/or light chain antibody sequence is from a first species and a second portion of the heavy and/or light chain antibody sequence is from a second species. Chimeric antibodies of interest herein include “primatized” antibodies comprising variable domain antigen-binding sequences derived from a non-human primate (e.g. Old-World Monkey, such as baboon, rhesus or cynomolgus monkey) and human constant region sequences. “Humanized” antibodies are chimeric antibodies that contain a sequence derived from non-human antibodies. For the most part, humanized antibodies are human antibodies (recipient antibody) in which residues from a hypervariable region of the recipient are replaced by residues from a hypervariable region [or complementarity determining region (CDR)] of a non-human species (donor antibody) such as mouse, rat, rabbit, chicken or non-human primate, having the desired specificity, affinity, and activity. In most instances residues of the human (recipient) antibody outside of the CDR; i.e. in the framework region (FR), are additionally replaced by corresponding non-human residues. Furthermore, humanized antibodies may comprise residues that are not found in the recipient antibody or in the donor antibody. These modifications are made to further refine antibody properties. Humanization reduces the immunogenicity of non-human antibodies in humans, thus facilitating the application of antibodies to the treatment of human disease. Humanized antibodies and several different technologies to generate them are well known in the art. The term “antibody” also refers to human antibodies, which can be generated as an alternative to humanization. For example, it is possible to produce transgenic animals (e.g., mice) that are capable, upon immunization, of producing a full repertoire of human antibodies in the absence of production of endogenous murine antibodies. Other methods for obtaining human antibodies/antibody fragments in vitro are based on display technologies such as phage display or ribosome display technology, wherein recombinant DNA libraries are used that are either generated at least in part artificially or from immunoglobulin variable (V) domain gene repertoires of donors. Phage and ribosome display technologies for generating human antibodies are well known in the art. Human antibodies may also be generated from isolated human B cells that are ex vivo immunized with an antigen of interest and subsequently fused to generate hybridomas which can then be screened for the optimal human antibody. The term “antibody” refers to both glycosylated and aglycosylated antibodies. Furthermore, the term “antibody” as used herein not only refers to full-length antibodies, but also refers to antibody fragments, more particularly to antigen-binding fragments thereof. A fragment of an antibody comprises at least one heavy or light chain immunoglobulin domain as known in the art and binds to one or more antigen(s). Examples of antibody fragments according to the invention include a Fab, modified Fab, Fab′, modified Fab′, F(ab′)₂, Fv, Fab-Fv, Fab-dsFv, Fab-Fv-Fv, scFv and Bis-scFv fragment. Said fragment can also be a diabody, tribody, triabody, tetrabody, minibody, single domain antibody (dAb) such as sdAb, VL, VH, VHH or camelid antibody (e.g. from camels or llamas such as a Nanobody™) and VNAR fragment. An antigen-binding fragment according to the invention can also comprise a Fab linked to one or two scFvs or dsscFvs, each scFv or dsscFv binding the same or a different target (e.g., one scFv or dsscFv binding a therapeutic target and one scFv or dsscFv that increases half-life by binding, for instance, albumin). Exemplary of such antibody fragments are FabdsscFv (also referred to as BYbe®) or Fab-(dsscFv) 2 (also referred to as TrYbe®, see e.g. WO2015/197772). Antibody molecules as defined above, including antigen-binding fragments thereof, are known in the art.

The term “epitope” refers to a region of an antigen that is bound by an antibody. Epitopes may be defined as structural or functional. Functional epitopes are generally a subset of the structural epitopes and have those residues that directly contribute to the affinity of the interaction. Epitopes may also be conformational, that is, composed of non-linear amino acids. In certain embodiments, epitopes may include determinants that are chemically active surface groupings of molecules such as amino acids, sugar side chains, phosphoryl groups, or sulfonyl groups, and, in certain embodiments, may have specific three-dimensional structural characteristics, and/or specific charge characteristics.

The term “treating” or “treatment” of a disease state includes: (i) inhibiting the disease state, i.e. arresting the development of the disease state or its clinical symptoms, or (ii) relieving the disease state, i.e. causing temporary or permanent regression of the disease state or its clinical symptoms.

The term “preventing” or “prevention” of a disease state includes causing the clinical symptoms of the disease state not to develop in a subject that may be exposed to or predisposed to the disease state, but does not yet experience or display symptoms of the disease state.

### DETAILED DESCRIPTION OF THE INVENTION

The invention is based on the finding from the inventor that total TG2 mRNA and total TG2 protein were increased in IPF patient lung tissues. TG2 is associated with increased collagen levels which correlate with decreased lung function. It was also a finding that mice lacking TG2 (referred to as knockout or KO mice) were protected from the development of interstitial lung fibrosis and subsequently loss of lung function after provocation with bleomycin (a drug that causes pulmonary fibrosis and is a widely used animal model of pulmonary fibrosis). The inventors were then able to surprisingly demonstrate that not only anti-TG2 antibodies can attenuate ECM deposition by human primary IPF cells in invitro assays, but also that an anti-TG2 antibody (inhibiting extracellular protein crosslinking activity of TG2) significantly attenuated pulmonary fibrosis in vivo (in a rabbit lung silicosis model) when given preventively, and arrested further progression of fibrosis when given starting 28 days after initiation of fibrosis. Further, it was surprisingly shown by the inventors that TG2 was upregulated in post-mortem lung samples from patients who have died from COVID-19 infection (caused by the SARS-Cov2 virus), with evidence of extensive matrix deposition.

The main object of the present invention is an anti-transglutaminase 2 (anti-TG2) antibody for use in the treatment of progressive chronic interstitial lung disease or in the prevention of the development of progressive chronic interstitial lung disease. For instance, the anti-TG2 antibody for use according to the invention can comprises the following sequences:

(i)
(LCDR1; SEQ ID NO. 1)
KASQDINSYLT;
(LCDR2; SEQ ID NO. 2)
LVNRLVD;
(LCDR3; SEQ ID NO. 3)
LQYDDFPYT;
(HCDR1; SEQ ID NO. 4)
THAMS;
(HCDR2; SEQ ID NO. 5)
TISSGGRSTYYPDSVKG;
and
(HCDR3; SEQ ID NO. 6)
LISTY;
or
(ii)
(LCDR1; SEQ ID NO. 1)
KASQDINSYLT;
(LCDR2; SEQ ID NO. 7)
LTNRLMD;
(LCDR3; SEQ ID NO. 8)
LQYVDFPYT;
(HCDR1; SEQ ID NO. 9)
SSAMS;
(HCDR2; SEQ ID NO. 5)
TISSGGRSTYYPDSVKG;
(HCDR3; SEQ ID NO. 10)
and
LISPY;
(iii)
(LCDR1; SEQ ID NO. 1)
ASQDINSYLT;
(LCDR2; SEQ ID NO. 11)
RTNRLFD;
(LCDR3; SEQ ID NO. 3)
LQYDDFPYT;
(HCDR1)
SSAMS;
(HCDR2; SEQ ID NO. 9)
TISVGGGKTYYPDSVKG;
and
(HCDR3; SEQ ID NO. 12)
LISLY,
or
(iv) compete with an antibody according to any
one of (i) to (iii).

The invention also provides a method for treating or for the prevention of the development of progressive chronic interstitial lung disease comprising administering a therapeutically effective amount of an anti-transglutaminase 2 (anti-TG2) antibody. For instance, the anti-TG2 that can be administered in such a method can comprises the following sequences:

(i)
(LCDR1; SEQ ID NO. 1)
KASQDINSYLT;
(LCDR2; SEQ ID NO. 2)
LVNRLVD;
(LCDR3; SEQ ID NO. 3)
LQYDDFPYT;
(HCDR1; SEQ ID NO. 4)
THAMS;
(HCDR2; SEQ ID NO. 5)
TISSGGRSTYYPDSVKG;
and
(HCDR3; SEQ ID NO. 6)
LISTY;
or
(ii)
(LCDR1; SEQ ID NO. 1)
KASQDINSYLT;
(LCDR2; SEQ ID NO. 7)
LTNRLMD;
(LCDR3; SEQ ID NO. 8)
LQYVDFPYT;
(HCDR1; SEQ ID NO. 9)
SSAMS;
(HCDR2; SEQ ID NO. 5)
TISSGGRSTYYPDSVKG;
(HCDR3; SEQ ID NO. 10)
and
LISPY;
(iii)
(LCDR1; SEQ ID NO. 1)
ASQDINSYLT;
(LCDR2; SEQ ID NO. 11)
RTNRLFD;
(LCDR3; SEQ ID NO. 3)
LQYDDFPYT;
(HCDR1)
SSAMS;
(HCDR2; SEQ ID NO. 9)
TISVGGGKTYYPDSVKG;
and
(HCDR3; SEQ ID NO. 12)
LISLY,
or
(iv) compete with an antibody according to any
one of (i) to (iii).

Also described is the use of an anti-transglutaminase 2 (anti-TG2) antibody for the manufacturing of a medicament in the treatment of a progressive chronic interstitial lung disease or for the preventing the development of progressive chronic interstitial lung disease. For instance, the anti-TG2 for use according to the invention can comprises the following sequences:

(i)
(LCDR1; SEQ ID NO. 1)
KASQDINSYLT;
(LCDR2; SEQ ID NO. 2)
LVNRLVD;
(LCDR3; SEQ ID NO. 3)
LQYDDFPYT;
(HCDR1; SEQ ID NO. 4)
THAMS;
(HCDR2; SEQ ID NO. 5)
TISSGGRSTYYPDSVKG;
and
(HCDR3; SEQ ID NO. 6)
LISTY;
or
(ii)
(LCDR1; SEQ ID NO. 1)
KASQDINSYLT;
(LCDR2; SEQ ID NO. 7)
LTNRLMD;
(LCDR3; SEQ ID NO. 8)
LQYVDFPYT;
(HCDR1; SEQ ID NO. 9)
SSAMS;
(HCDR2; SEQ ID NO. 5)
TISSGGRSTYYPDSVKG;
(HCDR3; SEQ ID NO. 10)
and
LISPY;
(iii)
(LCDR1; SEQ ID NO. 1)
ASQDINSYLT;
(LCDR2; SEQ ID NO. 11)
RTNRLFD;
(LCDR3; SEQ ID NO. 3)
LQYDDFPYT;
(HCDR1)
SSAMS;
(HCDR2; SEQ ID NO. 9)
TISVGGGKTYYPDSVKG;
and
(HCDR3; SEQ ID NO. 12)
LISLY,
or
(iv) compete with an antibody according to any
one of (i) to (iii).

Another object of the present invention is an anti-transglutaminase 2 (anti-TG2) antibody for use in the treatment of a subject having a lung fibrosis associated with COVID infection or in the prevention of the development of a lung fibrosis in a subject suffering from COVID infection. For instance, the anti-TG2 antibody for use according to the invention can comprises the following sequences:

(i)
(LCDR1; SEQ ID NO. 1)
KASQDINSYLT;
(LCDR2; SEQ ID NO. 2)
LVNRLVD;
(LCDR3; SEQ ID NO. 3)
LQYDDFPYT;
(HCDR1; SEQ ID NO. 4)
THAMS;
(HCDR2; SEQ ID NO. 5)
TISSGGRSTYYPDSVKG;
and
(HCDR3; SEQ ID NO. 6)
LISTY;
or
(ii)
(LCDR1; SEQ ID NO. 1)
KASQDINSYLT;
(LCDR2; SEQ ID NO. 7)
LTNRLMD;
(LCDR3; SEQ ID NO. 8)
LQYVDFPYT;
(HCDR1; SEQ ID NO. 9)
SSAMS;
(HCDR2; SEQ ID NO. 5)
TISSGGRSTYYPDSVKG;
(HCDR3; SEQ ID NO. 10)
and
LISPY;
(iii)
(LCDR1; SEQ ID NO. 1)
ASQDINSYLT;
(LCDR2; SEQ ID NO. 11)
RTNRLFD;
(LCDR3; SEQ ID NO. 3)
LQYDDFPYT;
(HCDR1)
SSAMS;
(HCDR2; SEQ ID NO. 9)
TISVGGGKTYYPDSVKG;
and
(HCDR3; SEQ ID NO. 12)
LISLY,
or
(iv) compete with an antibody according to any
one of (i) to (iii).

The invention also provides a method for treating a patient having a lung fibrosis associated with COVID infection or for preventing the development a lung fibrosis in a patient suffering from COVID infection, comprising the step of administering a therapeutically effective amount of an anti-transglutaminase 2 (anti-TG2) antibody. For instance, the anti-TG2 that can be administered in such a method can comprises the following sequences:

(i)
(LCDR1; SEQ ID NO. 1)
KASQDINSYLT;
(LCDR2; SEQ ID NO. 2)
LVNRLVD;
(LCDR3; SEQ ID NO. 3)
LQYDDFPYT;
(HCDR1; SEQ ID NO. 4)
THAMS;
(HCDR2; SEQ ID NO. 5)
TISSGGRSTYYPDSVKG;
and
(HCDR3; SEQ ID NO. 6)
LISTY;
or
(ii)
(LCDR1; SEQ ID NO. 1)
KASQDINSYLT;
(LCDR2; SEQ ID NO. 7)
LTNRLMD;
(LCDR3; SEQ ID NO. 8)
LQYVDFPYT;
(HCDR1; SEQ ID NO. 9)
SSAMS;
(HCDR2; SEQ ID NO. 5)
TISSGGRSTYYPDSVKG;
(HCDR3; SEQ ID NO. 10)
and
LISPY;
(iii)
(LCDR1; SEQ ID NO. 1)
ASQDINSYLT;
(LCDR2; SEQ ID NO. 11)
RTNRLFD;
(LCDR3; SEQ ID NO. 3)
LQYDDFPYT;
(HCDR1)
SSAMS;
(HCDR2; SEQ ID NO. 9)
TISVGGGKTYYPDSVKG;
and
(HCDR3; SEQ ID NO. 12)
LISLY,
or
(iv) compete with an antibody according to any
one of (i) to (iii).

Also described is the use of an anti-transglutaminase 2 (anti-TG2) antibody for the manufacturing of a medicament in the treatment of a subject having a lung fibrosis associated with COVID infection or the prevention of the development a lung fibrosis in a subject suffering from COVID infection. For instance, the anti-TG2 for use according to the invention can comprises the following sequences:

(i)
(LCDR1; SEQ ID NO. 1)
KASQDINSYLT;
(LCDR2; SEQ ID NO. 2)
LVNRLVD;
(LCDR3; SEQ ID NO. 3)
LQYDDFPYT;
(HCDR1; SEQ ID NO. 4)
THAMS;
(HCDR2; SEQ ID NO. 5)
TISSGGRSTYYPDSVKG;
and
(HCDR3; SEQ ID NO. 6)
LISTY;
or
(ii)
(LCDR1; SEQ ID NO. 1)
KASQDINSYLT;
(LCDR2; SEQ ID NO. 7)
LTNRLMD;
(LCDR3; SEQ ID NO. 8)
LQYVDFPYT;
(HCDR1; SEQ ID NO. 9)
SSAMS;
(HCDR2; SEQ ID NO. 5)
TISSGGRSTYYPDSVKG;
(HCDR3; SEQ ID NO. 10)
and
LISPY;
(iii)
(LCDR1; SEQ ID NO. 1)
ASQDINSYLT;
(LCDR2; SEQ ID NO. 11)
RTNRLFD;
(LCDR3; SEQ ID NO. 3)
LQYDDFPYT;
(HCDR1)
SSAMS;
(HCDR2; SEQ ID NO. 9)
TISVGGGKTYYPDSVKG;
and
(HCDR3; SEQ ID NO. 12)
LISLY,
or
(iv) compete with an antibody according to any
one of (i) to (iii).

In the context of the present invention as a whole, the chronic progressive lung fibrotic disease is characterised by an increase of a marker in a subject's sample, wherein the marker is for instance any one of TG2 activity, TG2 expression (such as increase of mRNA encoding TG2 or of TG2 antigen), export of TG2 or any combination thereof, and wherein the subject's sample is a cell or a tissue associated with said disease (e.g. a lung cell or a lung tissue). Said increase of the marker may be determined by any means in cells/tissues associated with said disease. An increase of a marker in one subject's sample is typically determined by comparison of the level of said marker in the subject's sample to the level of the same marker in normal cells of the same tissue type (i.e. basal level; e.g. basal TG2 activity, basal expression level (mRNA level and/or protein level) and/or basal level of TG2 export). A subject's sample having a level of at least one marker equal or higher than 10%, equal or higher than 15%, equal or higher than 20%, equal or higher than 25% or even equal or higher than 30% compared to the basal level for said marker will be consider as presenting an increase of said marker. For example, an increase of TG2 expression (alternatively called TG2 overexpression) can be determined via determination of the amount of TG2 mRNA in lung cells of a patient. Chronic progressive lung fibrotic cells/tissues may thus be characterised for instance by an increased amount (representing overexpression) of TG2 mRNA in lung cells of a subject, compared with normal cells from the same tissue type. The expression of TG2 mRNA may be increased by any amount, such as equal or higher than 10%, equal or higher than 15%, equal or higher than 20%, equal or higher than 25% or even equal or higher than 30% compared to the basal level. The amount of mRNA can be measured using any known methods such as quantitative reverse transcription polymerase chain reaction (qRT-PCR), real time qRT-PCR, quantigene assay (Affymetrix/Thermo Fisher), by northern blotting or using microarrays, RNA sequencing and various types of in situ hybridisation (e.g. RNAscope). Alternatively, overexpression can be determined via determination of the amount of TG2 antigen in lung cells of a patient. The chronic progressive lung fibrotic cells may thus be characterised for instance by an increased amount (representing overexpression) of TG2 protein (or TG2 antigen) in lung cells of a subject, such as compared with normal cells of the same tissue type. The expression of TG2 protein may be increased by any amount, such as equal or higher than 10%, equal or higher than 15%, equal or higher than 20%, equal or higher than 25% or even equal or higher than 30% % compared to the basal level. The amount of protein can be measured using any known methods such as immunohistochemistry, western blotting, mass spectrometry or fluorescence-activated cell sorting (FACS), including by use of an anti-TG2 antibody of the invention. The thresholds for determining expression may vary depending on the techniques that are used and may be validated against immunohistochemistry scores. Alternatively, the chronic progressive lung fibrotic cells may be characterised by an increase of the TG2 activity in lung cells of a subject, compared with normal cells of the same tissue type. TG2 activity may be increased by any amount, such as equal or higher than 10%, equal or higher than 15%, equal or higher than 20%, equal or higher than 25% or even equal or higher than 30% % compared to the basal level. TG2 activity can be measured using any known methods such as via cryo biopsy (TG ISA), in exhaled breath condensates (EBC) or bronchoalveolar lavage fluid (BALF).

The anti-TG2 antibodies for use, method for treating or for preventing, or use of anti-TG2 according to the invention, e.g. for treating or preventing progressive chronic interstitial lung disease in a subject or for treating/preventing lung fibrosis associated with COVID infection, may thus comprise the steps of (a) measuring TG2 expression, TG2 activity or TG2 export in a sample (e.g. lung cells) from the subject, (b) comparing the result of the measure obtained from a) to the corresponding measure in a normal cell/tissue (such as lung cells), and c) if an increase of expression (i.e. overexpression of TG2), an increase of activity or an increase of export is observed, administering to the patient an anti-TG2 antibody, thereby treating or preventing the progressive chronic interstitial lung disease or the lung fibrosis associated with COVID infection. TG2 expression that is measured in step a) can be the mRNA or protein amount, and the increase can be any increase of expression as discussed above. The corresponding measure in a normal cell/tissue does not need to be obtained each time a comparison is to be made. Said corresponding measure can be obtained any time before the comparison is to be made and can be the average TG2 expression or TG2 activity in a normal cell/tissue.

In the context of the invention as a whole, the progressive chronic interstitial lung disease is selected from the group consisting of Idiopathic pulmonary fibrosis (IPF), Desquamative interstitial pneumonia (DIP), Acute interstitial pneumonia (AIP; alternatively known as Hamman-Rich syndrome), Hypersensitivity pneumonitis (HSP), Nonspecific interstitial pneumonia (NSIP), Respiratory bronchiolitis-associated interstitial lung disease (RB-ILD), Cryptogenic organizing pneumonia (COP; alternatively named Bronchiolitis Obliterans Organizing Pneumonia or BOOP), sarcoidosis, asbestosis and Lymphoid interstitial pneumonia (LIP). Preferably, the progressive chronic interstitial lung disease is selected from the group consisting of Idiopathic pulmonary fibrosis (IPF).

In the context of the invention as a whole, the anti-TG2 antibody preferably binds to an epitope within the core region of transglutaminase type 2 (TG2) and inhibits TG2 activity, wherein said core region consists of amino acids 143 to 473 of TG2 (e.g. of SEQ ID No. 41), and wherein the TG2 activity that is inhibited is the TG2 cross-linking of lysine and glutamine with N-ε(γ-glutamyl) lysine isopeptide bonds. Even preferably, the antibody binds to region comprising or consisting of amino acids 304 to 326 of TG2 (e.g. of SEQ ID No. 41) or part of this region. Said antibody can comprise or consist of an intact antibody. Alternatively it can comprise or consist of an antigen-binding fragment such as (but not limited to): an Fv fragment (for example a single chain Fv fragment or a disulphide-bonded Fv fragment); a Fab fragment; and a Fab-like fragment (for example an Fab′ fragment or an F (ab) 2 fragment), single domain antibody (or any other fragments as herein defined or known by the skilled person). Preferably, the anti-TG2 antibody to be used according to the invention as a whole (see also Table A):
a) comprises 6 CDRs selected from the group consisting of:

(i)
(LCDR1; SEQ ID NO. 1)
KASQDINSYLT;
(LCDR2; SEQ ID NO. 2)
LVNRLVD;
(LCDR3; SEQ ID NO. 3)
LQYDDFPYT;
(HCDR1; SEQ ID NO. 4)
THAMS;
(HCDR2; SEQ ID NO. 5)
TISSGGRSTYYPDSVKG;
and
(HCDR3; SEQ ID NO. 6)
LISTY;
or
(ii)
(LCDR1; SEQ ID NO. 1)
KASQDINSYLT;
(LCDR2; SEQ ID NO. 7)
LTNRLMD;
(LCDR3; SEQ ID NO. 8)
LQYVDFPYT;
(HCDR1; SEQ ID NO. 9)
SSAMS;
(HCDR2; SEQ ID NO. 5)
TISSGGRSTYYPDSVKG;
(HCDR3; SEQ ID NO. 10)
and
LISPY;
(iii)
(LCDR1; SEQ ID NO. 1)
ASQDINSYLT;
(LCDR2; SEQ ID NO. 11)
RTNRLFD;
(LCDR3; SEQ ID NO. 3)
LQYDDFPYT;
(HCDR1)
SSAMS;
(HCDR2; SEQ ID NO. 9)
TISVGGGKTYYPDSVKG;
and
(HCDR3; SEQ ID NO. 12)
LISLY,
or
(iv) compete with an antibody according to any
one of (i) to (iii).

b) comprises a light chain variable domain having the sequence as defined in any one of SEQ ID NO: 13 to SEQ ID No. 27 and a heavy chain variable domain having the sequence as defined in any one of SEQ ID NO: 28 to SEQ ID No. 40,
c) comprises a light chain variable domain having at least 80% identity or similarity, preferably at least 90% identity or similarity, or preferably at least 95% identity or similarity to the sequence as defined in any one of SEQ ID NO: 13 to SEQ ID No. 27 and a heavy chain variable domain having at least 80% identity or similarity, preferably at least 90% identity or similarity, or preferably at least 95% identity or similarity to the sequence as defined in any one of SEQ ID NO: 28 to SEQ ID No. 40,
d) compete for binding to an epitope comprising or consisting of amino acids 304 to 326 of TG2 (e.g. of SEQ ID NO. 41) or part of this region with an antibody as defined in a), b) or c) above.

TABLE A
Amino acid sequences

| SEQ ID | Amino acid sequence |
|---|---|
| 1 | KASQDINSYLT |
| 2 | LVNRLVD |
| 3 | LQYDDFPYT |
| 4 | THAMS |
| 5 | TISSGGRSTYYPDSVKG |
| 6 | LISTY |
| 7 | LTNRLMD |
| 8 | LQYVDFPYT |
| 9 | SSAMS |
| 10 | LISPY |
| 11 | RTNRLFD |
| 12 | LISLY |
| 13 | DIQMTQTPSSMYASLGERVTITCKASQDINSYLTWFQQKPGKSPKTLIYRTNRLFD |
| | GVPSRFSGSGSGQDFFLTISSLEYEDMGIYYCLQYDDFPYTFGGGTKLEIK |
| 14 | DIKMTQSPSSMYASLGERVTITCKASQDINSYLTWFQQKPGKSPKTLIYRTNRLFD |
| | GVPSRFSGSGSGQDFFLTISSLEYEDMGIYYCLQYDDFPYTFGGGTKLEIK |
| 15 | EIVLTQSPSSMYASLGERVTITCKASQDINSYLTWFQQKPGKSPKTLIYRTNRLFDG |
| | VPSRFSGSGSGQDFFLTISSLEYEDMGIYYCLQYDDFPYTFGGGTKLEIK |
| 16 | DIQMTQSPSSMYASLGERVTITCKASQDINSYLTWFQQKPGKSPKTLIYRTNRLFD |
| | GVPSRFSGSGSGQDFFLTISSLEYEDMGIYYCLQYDDFPYTFGGGTKLEIK |
| 17 | EIVLTQSPSSLSASVGDRVTITCKASQDINSYLTWYQQKPGKAPKLLIYRTNRLFDG |
| | VPSRFSGSGSGTDFFFTISSLQPEDFGTYYCLQYDDFPYTFGGGTKLEIK |
| 18 | DIQMTQSPSSLSASVGDRVTITCKASQDINSYLTWFQQKPGKAPKSLIYRTNRLFD |
| | GVPSRFSGSGSGTDFFLTISSLQPEDFATYYCLQYDDFPYTFGQGTKVEIK |
| 19 | AIKMTQSPSSMYASLGERVIITCKASQDINSYLTWFQQKPGKSPKTLIYLTNRLMDG |
| | VPSRFSGSGSGQEFLLTISGLEHEDMGIYYCLQYVDFPYTFGGGTKLEIK |
| 20 | DIQMTQSPSSLSASVGDRVTITCKASQDINSYLTWFQQKPGKAPKSLIYLTNRLMD |
| | GVPSRFSGSGSGTDFFLTISSLQPEDFATYYCLQYVDFPYTFGQGTKVEIK |
| 21 | DIKMTQSPSSLSASVGDRVTITCKASQDINSYLTWFQQKPGKAPKTLIYLTNRLMD |
| | GVPSRFSGSGSGQEFLLTISSLQPEDFATYYCLQYVDFPYTFGQGTKVEIK |
| 22 | DITMTQSPSSIYASLGERVTITCKASQDINSYLTWFQQKPGKSPKILIYLVNRLVDGV |
| | PSRFSGSGSGQDYALTISSLEYEDMGIYYCLQYDDFPYTFGGGTKLEIK |
| 23 | DIQMTQSPSSLSASVGDRVTITCKASQDINSYLTWFQQKPGKAPKSLIYLVNRLVD |
| | GVPSRFSGSGSGTDFFLTISSLQPEDFATYYCLQYDDFPYTFGQGTKVEIK |
| 24 | DITMTQSPSSLSASVGDRVTITCKASQDINSYLTWFQQKPGKAPKILIYLVNRLVDGVPS |
| | RFSGSGSGQDYALTISSLQPEDFATYYCLQYDDFPYTFGQGTKVEIK |
| 25 | AIKMTQTPSSVSAAVGGTVTISCKASQDINSYLTWFQQKPGQPPKTLIYLTNRLMDGVP |
| | SRFSGSGSGQEFLLTISGLECDDAATYYCLQYVDFPYTFGGGTKVVVK |
| 26 | DVVNTQTPLTLSVTFGQPASISCKSSQSLLYDNGKTYLHWLFQRPGQSPRRLIYLVSKL |
| | DSGVPDRFTGSGSGTDFTLKISRVEAEDLGVYYCVQGTHFPYTFGGGTKLEIK |
| 27 | QIVLTQSPAIMSASPGEKVTMTCSASSSVSYMYWYQQKPGSSPRLLIYDTSNLASGVP |
| | VRFSGSGSGTSYSLTISRMEAEDAATFYCQQWSSSPLTFGAGTKLELK |
| 28 | EVQLEESGGGLVKPGGSLKLSCAASGFTLSSSAMSWVRQTPDRRLEWVATISVG |
| | GGKTYYPDSVKGRFTISRDNAKNTLYLQMNSLRSEDTAMYYCAKLISLYWGQGTT |
| | LTVSS |
| 29 | EVQLVESGGGLVKPGGSLKLSCAASGFTLSSSAMSWVRQTPDRRLEWVATISVG |
| | GGKTYYPDSVKGRFTISRDNAKNTLYLQMNSLRSEDTAMYYCAKLISLYWGQGTT |
| | LTVSS |
| 30 | EVQLQESGGGLVKPGGSLKLSCAASGFTLSSSAMSWVRQTPDRRLEWVATISVG |
| | GGKTYYPDSVKGRFTISRDNAKNTLYLQMNSLRSEDTAMYYCAKLISLYWGQGTT |
| | LTVSS |
| 31 | EVQLLESGGGLVQPGGSLRLSCAASGFTFSSSAMSWVRQAPGKGLEWVSTISVG |
| | GGKTYYPDSVKGRFTISRDNSKNTLYLQMNSLRAEDTAVYYCAKLISLYWGQGTL |
| | VTVSS |
| 32 | AVQLVESGGGLVKPGGSLKLSCAASGIIFSSSAMSWVRQTPEKRLEWVATISSGG |
| | RSTYYPDSVKGRFTVSRDSAKNTLYLQMDSLRSEDTAIYYCAKLISPYWGQGTTLT |
| | VSS |
| 33 | EVQLLESGGGLVQPGGSLRLSCAASGFTFSSSAMSWVRQAPGKGLEWVSTISSG |
| | GRSTYYPDSVKGRFTISRDNSKNTLYLQMNSLRAEDTAVYYCAKLISPYWGQGTL |
| | VTVSS |
| 34 | EVQLLESGGGLVQPGGSLRLSCAASGIIFSSSAMSWVRQAPGKGLEWVATISSGG |
| | RSTYYPDSVKGRFTVSRDSSKNTLYLQMNSLRAEDTAVYYCAKLISPYWGQGTLV |
| | TVSS |
| 35 | EVOLVESGGGLVKPGGSLKLSCAASGFTLSTHAMSWVRQTPEKRLEWVATISSG |
| | GRSTYYPDSVKGRFTISRDNVKNTLYLQLSSLRSEDTAVYFCARLISTYWGQGTTL |
| | TVSS |
| 36 | EVQLLESGGGLVQPGGSLRLSCAASGFTFSTHAMSWVRQAPGKGLEWVSTISSG |
| | GRSTYYPDSVKGRFTISRDNSKNTLYLQMNSLRAEDTAVYYCAKLISTYWGQGTL |
| | VTVSS |
| 37 | EVQLLESGGGLVQPGGSLRLSCAASGFTLSTHAMSWVRQAPGKGLEWVATISSGGR |
| | STYYPDSVKGRFTISRDNSKNTLYLQMNSLRAEDTAVYFCARLISTYWGQGTLVTVSS |
| 38 | EVQLLESGGRLVTPGGSLTLTCAASGIIFSSSAMSWVRQAPGKGLEWVATISSGGRST |
| | YYPDSVKGRFTVSRDSSKNTLYLQMNSLRAEDTAVYYCAKLISPYWGQGTLVTVSS |
| 39 | QIQLVQSGPELKKPGETVKISCKASGYTFTTYGNTWVKQAPGKGLKWMGWINTSSGV |
| | PTYADDFKGRFAFSLETSASTAYLQINNLKSEDTATYFCARPEVAYWGQGTLVTVSA |
| 40 | QVTLKESGPGILQPSQTLSLTCSFSGFSLSTSGMGVSWIRQPSGKGLEWLAHIYWDDD |
| | KRYNPSLKSRLTISKDTSSNQVFLKITSVDTADTATYYCARSGTTAPFAYWGQGTLVTV |
| | SA |
| 41 | AEELVLERCDLELETNGRDHHTADLCREKLVVRRGQPFWLTLHFEGRNYEASVDSLTF |
| | SVVTGPAPSQEAGTKARFPLRDAVEEGDWTATVVDQQDCTLSLQLTTPANAPIGLYRL |
| | SLEASTGYQGSSFVLGHFILLFNAWCPADAVYLDSEEERQEYVLTQQGFIYQGSAKFIK |
| | NIPWNFGQFEDGILDICLILLDVNPKFLKNAGRDCSRRSSPVYVGRVVSGMVNCNDDQ |
| | GVLLGRWDNNYGDGVSPMSWIGSVDILRRWKNHGCQRVKYGQCWVFAAVACTVLR |
| | CLGIPTRVVTNYNSAHDQNSNLLIEYFRNEFGEIQGDKSEMIWNFHCWVESWMTRPDL |
| | QPGYEGWQALDPTPQEKSEGTYCCGPVPVRAIKEGDLSTKYDAPFVFAEVNADVVD |
| | WIQQDDGSVHKSINRSLIVGLKISTKSVGRDEREDITHTYKYPEGSSEEREAFTRANHL |
| | NKLAEKEETGMAMRIRVGQSMNMGSDFDVFAHITNNTAEEYVCRLLLCARTVSYNGIL |
| | GPECGTKYLLNLNLEPFSEKSVPLCILYEKYRDCLTESNLIKVRALLVEPVINSYLLAERD |
| | LYLENPEIKIRILGEPKQKRKLVAEVSLQNPLPVALEGCTFTVEGAGLTEEQKTVEIPDP |
| | VEAGEEVKVRMDLLPLHMGLHKLVVNFESDKLKAVKGFRNVIIGPA |

One can easily determine whether an antibody binds to the same epitope as, or competes for binding with, another antibody by using routine methods known in the art. For example, to determine if a test antibody binds to the same epitope as a reference antibody of the invention, the reference antibody is allowed to bind to a protein or peptide under saturating conditions. Next, the ability of a test antibody to bind to the protein or peptide is assessed. If the test antibody is able to bind to the protein or peptide following saturation binding with the reference antibody, it can be concluded that the test antibody binds to a different epitope than the reference antibody. On the other hand, if the test antibody is not able to bind to protein or peptide following saturation binding with the reference antibody, then the test antibody may bind to the same epitope as the epitope bound by the reference antibody of the invention. To determine if an antibody competes for binding with a reference antibody, the above-described binding methodology is performed in two orientations. In a first orientation, the reference antibody is allowed to bind to a protein/peptide under saturating conditions followed by assessment of binding of the test antibody to the protein/peptide molecule. In a second orientation, the test antibody is allowed to bind to the protein/peptide under saturating conditions followed by assessment of binding of the reference antibody to the protein/peptide. If, in both orientations, only the first (saturating) antibody is capable of binding to the protein/peptide, then it is concluded that the test antibody and the reference antibody compete for binding to the protein/peptide. As will be appreciated by the skilled person, an antibody that competes for binding with a reference antibody may not necessarily bind to the identical epitope as the reference antibody but may sterically block binding of the reference antibody by binding an overlapping or adjacent epitope.

Two antibodies bind to the same or overlapping epitope if each competitively inhibits (blocks) binding of the other to the antigen. That is, a 1-, 5-, 10-, 20- or 100-fold excess of one antibody inhibits binding of the other by at least 50%, 75%, 90% or even 99% as measured in a competitive binding assay. Alternatively, two antibodies have the same epitope if essentially all amino acid mutations in the antigen that reduce or eliminate binding of one antibody reduce or eliminate binding of the other. Two antibodies have overlapping epitopes if some amino acid mutations that reduce or eliminate binding of one antibody reduce or eliminate binding of the other.

Additional routine experimentation (e.g., peptide mutation and binding analyses) can then be carried out to confirm whether the observed lack of binding of the test antibody is in fact due to binding to the same epitope as the reference antibody or if steric blocking (or another phenomenon) is responsible for the lack of observed binding. Experiments of this sort can be performed using ELISA, RIA, surface plasmon resonance, flow cytometry or any other quantitative or qualitative antibody-binding assay available in the art.

Any subject may be treated in accordance with the invention. The subject is preferably human. However, the subject may be another mammalian animal, such as a non-human primate, a horse, a cow, a sheep, a pig, a dog, a cat, a rabbit, a rat, a mouse, a guinea pig or a hamster. Alternatively, the term patient can be used indifferently instead of subject.

Any anti-TG2 antibody according to the invention may be incorporated into pharmaceutical compositions suitable for administration to a subject in any way, such as (but not limited to) topically, intra nasally, intradermally, intravenously, subcutaneously or intramuscularly. Typically, the pharmaceutical composition comprises the anti-TG2 antibody and one or more pharmaceutically acceptable adjuvant(s) and/or carrier(s). Therefore, herein described is also a pharmaceutical composition for use in the treatment of progressive chronic interstitial lung disease, such as Idiopathic Pulmonary Fibrosis (IPF), wherein said pharmaceutical composition comprises an anti-TG2 antibody and one or more pharmaceutically acceptable adjuvant(s) and/or carrier(s). The pharmaceutical composition according to the invention can be part of a kit with instructions for use, including instructions and optionally a device for intravenous, subcutaneous or intramuscular administration to the individual in need thereof.

As used herein, “pharmaceutically acceptable carrier” includes any and all solvents, dispersion media, coatings, antibacterial and antifungal agents, isotonic and absorption delaying agents, and the like that are physiologically compatible and are suitable for administration to a subject for the methods and uses described herein. Examples of pharmaceutically acceptable carriers include one or more of water, saline, phosphate buffered saline, dextrose, glycerol, ethanol and the like, as well as combinations thereof. Depending on the route of administration or the type of formulation (such as liquid, freeze-dried or spray-dried formulation), isotonic agents can be incorporated, for example, sugars, polyalcohols such as mannitol, sorbitol, or sodium chloride in the composition. Pharmaceutically acceptable carriers may further comprise minor amounts of auxiliary substances such as wetting or emulsifying agents, preservatives or buffers, which enhance the shelf life or effectiveness of the antibody or antibody portion.

The pharmaceutical compositions according to the present invention may be in a variety of forms. These include, for example, liquid solutions (e.g., injectable and infusible solutions), dispersions or suspensions, powders and liposomes. The preferred form depends on the intended mode of administration and therapeutic application. Typical preferred compositions are in the form of injectable or infusible solutions, such as compositions similar to those used for passive immunization of humans with other antibodies.

A suitable dosage of an anti-TG2 antibody according to the present invention may be determined by a skilled medical practitioner. Actual dosage levels of the active ingredients in the pharmaceutical compositions of the present invention may be varied so as to obtain an amount of the active ingredient that is effective to achieve the desired therapeutic response for a particular patient, composition, and mode of administration, without being toxic to the patient. The selected dosage level will depend upon a variety of pharmacokinetic factors including the route of administration, the time of administration, the rate of excretion of the antibody, the duration of the treatment, other drugs, compounds and/or materials used in combination with the particular antibody, the age, sex, weight, condition, general health and prior medical history of the patient being treated.

A suitable dose may be, for example, in the range of from about 0.01 pg/kg to about 1000 mg/kg body weight, typically from about 0.1 pg/kg to about 100 mg/kg body weight, of the patient to be treated. Dosage regimens may be adjusted to provide the optimum desired response (e.g. a therapeutic response). For example, a single dose may be administered, or several divided doses may be administered over time. Dosage unit form as used herein refers to physically discrete units suited as unitary dosages for the subjects to be treated; each unit contains a predetermined quantity of active compound calculated to produce the desired therapeutic effect in association with the required pharmaceutical earner. Administration may be in single or multiple doses. Multiple doses may be administered via the same or different routes and to the same or different locations. In the context of the invention as a whole the anti-TG2 antibody may be co-administered with one or other more other therapeutic agents. Combined administration of two or more agents may be achieved in a number of different ways. Both may be administered together in a single composition, or they may be administered in separate compositions as part of a combined therapy. For example, the one may be administered before or separately, after or sequential, or concurrently or simultaneously with the other.

### DESCRIPTION OF THE FIGURES

FIG. 1: Collagen staining is elevated in lung biopsy from IPF patients. (A) Percent area of tissue section that was positively Picosirius red stained for dense collagen fibres associated with mature scar tissue. (B) Percent non-airway collagen within the total tissue section. Non-fibrotic (patients requiring lung biopsy that did not have ILD) were used as control samples (n=8). IPF/UIP samples were averaged together (n=9). *p<0.05 by unpaired, 2-sided t-tests.

FIG. 2: TG2 mRNA (RNAscope) is elevated in IPF patient biopsy. Formalin fixed paraffin embedded lung tissue samples were probed for Tgm2 expression using the in-situ hybridization technique RNAScope. Red/pink staining represented Tgm2 mRNA transcripts. (A): Total number of Tgm2 positive cells within a biopsy. (B): Tgm2 positive cells by intensity of staining. Staining intensity is represented as 1+(1-6 probes per cell), 2+(7-13 probes per cell) and 3+(≥14 probes per cell) (p<0.015). Only 2 of the 10 “non-fibrotic” biopsy analysed are shown as the others failed RNA quality QC checks.

FIG. 3: (A) TG2 antigen is elevated in IPF patient biopsy. TG2 immunohistochemistry staining on paraffin fixed sections from non-fibrotic and IPF samples were analysed and the percentage of stained area calculated by high content image analysis. Non-fibrotic (n=7) and IPF/UIP samples (n=10) were averaged. p=0.03 by unpaired, 2-sided t-tests. (B) Lung function measurements on patients, using forced vital capacity as percent predicted (FVC) (n=10). Linear regressions were performed between FVC and collagen (determined by PSR staining, see circles; P=0.0128, R2=0.5597 as well as by SHG, see triangles, P=0.0983, R2=0.3043).

FIG. 4: Knockout (KO) mouse is protected from collagen increase as measured by second harmonic generation microscopy; i.e. SHG in the bleomycin model of interstitial lung disease. (A): Percentage area of interstitial collagens in wildtype and TG2 KO mice. (B): Fold increase in lung interstitial collagen in wildtype and TG2 KO mice in response to bleomycin. (n=9-12).

FIG. 5: The TG2 knockout mouse does not have elevated mRNA (RT QPCR) for key interstitial collagens in the bleomycin model of interstitial lung disease. N=5 mice per group. ANOVA with Tukey post-test.

FIG. 6: TG2 knock out mice have preserved lung function in the bleomycin model of interstitial lung disease. (A): Resistance (Rrs=respiratory system resistance (Total); Rn=Newtonian (airway) resistance). (B): Compliance (Crs=dynamic compliance (compliance of the respiratory system); Cst=quasi-static compliance). (C): Pressure Volume Loop (K=curvature of the deflation limb of the PV loop; PV=pressure-volume). (D): Elastance (Ers=elastance of the respiratory system (Total); H=tissue elastance); ** P<0.01, *** P<0.001 by ANOVA with Tukey post-test.

FIG. 7: (A): Representative images from TGF β1 stimulated cells showing in corporation of 5-BP (i.e. Transglutaminase activity) when the label was added immediately after a scratch (left panel), after 1 hour (centre panel) and 3 hours (right panel) demonstrating activity is lost within the first hour post scratch. (B): Fibronectin and TG2 activity were quantified in the cell layer (away from the scratch) and at the scratch boundary in both non-fibrotic (NF) and fibrotic (F) fibroblasts with and without TGFβ1 treatment. 5-BP was added immediately after the scratch wound was introduced and the cells fixed after 1 hour. Each point represents staining in a single well. P-values as shown. (C): A monolayer of TGFβ1 treated lung fibroblasts was scratched and 5-BP added immediately afterwards as described above and treated with either vehicle (left panel) or TG2 inhibitory antibody 300 uM rbBB7 (right panel). rbBB7 completely inhibited incorporation of 5-BP as shown by the absence of staining.

FIG. 8: Rabbit lung silicosis study plan. New Zealand white rabbits either had saline or silica after a scratch delivered to the lower lobe of the right lung by bronchoscope. 6 experimental groups were defined. 5 animals (group 1) had just saline delivered and were left to run for 56 days as the “healthy control” group. 40 animals had silica delivered. 10 animals (group 2) were stopped after 28 days to be used as a measure of disease at the start of therapeutic dosing. 10 animals were treated with vehicle (group 3, n=5) or a control IgG rb922 (group 4, n=5) and run for 56 days as “untreated” controls. (The results from the vehicle and control IgG groups were essentially similar and the groups were combined into a single group of 10 untreated controls.) 10 animals were treated with the TG2 inhibitory rabbit antibody rbBB7 from day 28 to 56 (group 5) as a therapeutic dosing arm and 10 animals were treated from day 1 to day 56 with rbBB7 as a “prophylactic” dosing arm. rbBB7 was applied every 5 days. Terminal analysis consisted of TG2 activity (TG ISA), TG2 antigen (TG2 Ant), Picosirius red staining (PSR), Masson's Trichrome staining (MT), Second harmonic generation quantification of lung collagen (HG) and various messenger RNA (mRNA).

FIG. 9: rbBB7 serum exposure in rabbit silicosis model. Prophylactic (from day 1, plot A and B) and therapeutic (day 26 to 56, plot C and D) dosing were both performed in 2 separate runs and thus plotted individually.

FIG. 10: rbBB7 reduces total lung collagen when measured by Picrosirius red staining in Rabbit Silicosis model of ILD. Collagen was determined in the “parenchyma” (excluding large airways, which contain pre-existing collagen not related to the disease process) (A) and within active fibrotic lesions (B). Silica d28 and silica d56 groups are stopped on days 28 or 56 respectively & not treated with the pharmacological agent either receiving nothing (d28) or vehicle/control IgG (d56). Silica+rbBB7 (d28-d56) is the therapeutic dosing group receiving rbBB7 from day 28 to day 56. Silica+rbBB7 (day 1-56) is the prophylactic dosing group receiving rbBB7 from 1 day before silica infusion. Each point represents one rabbit. Statistics shown between groups are 1-way Students t-tests and demonstrate reduced fibrosis in both prophylactic and therapeutic arms. When analysed by ANOVA, the overall treatment effect was significant (p=0.002 for collagen in lesions and p=0.0037 for collagen in parenchyma).

FIG. 11: rbBB7 reduces total lung collagen when measured by second harmonic generation microscopy (SHG) in Rabbit Silicosis model of ILD. Silica d28 and silica d56 groups are stopped on days 28 or 56 respectively & not treated with the pharmacological agent either receiving nothing (d28) or vehicle/control IgG (d56). Silica+rbBB7 (d28-d56) is the therapeutic dosing group receiving rbBB7 from day 28 to day 56. Silica+rbBB7 (day 1-56) is the prophylactic dosing group receiving rbBB7 from 1 day before silica infusion. Each point represents one rabbit. Statistics shown between groups are 1-way Students t-tests and demonstrate reduced fibrosis in both prophylactic and therapeutic arms.

FIG. 12: rbBB7 blocks TG2 activity in the rabbit silicosis model. TG2 antigen (A) and extracellular TG in situ (ISA) activity (B) were measured in the study described in FIG. 11 at animal termination using high content image analysis of lung sections stained for TG2 antigen using immunofluorescence or TG2 activity by incorporation of fluorochrome labelled cadaverine respectively. Staining was quantified as percentage of the tissue areas defined by nuclear DAPI staining. *=P<0.05 by ANOVA. Data demonstrates no change in TG2 antigen levels, but a clear increase in TG activity in the silica groups that's is reduced in the therapeutic dosing arm, and significantly reduced in the prophylactic dosing group.

FIG. 13: TG2 mRNA (RNAscope) was upregulated in post-mortem lung samples from patients who have died from COVID-19 infection (caused by the SARS-Cov2 virus). Formalin fixed paraffin embedded lung tissue samples were probed for Tgm2 expression using the in-situ hybridization technique RNAScope. Black dots/staining represented Tgm2 mRNA transcripts. TG2 mRNA expression was compared to normal and IPF lung tissue

FIG. 14: Tgm2 Expression in Post Mortem Covid Lung. Tgm2 expression was assessed in lung tissue from normal, IPF and subjects who died as a result of COVID infection. Expression was calculated as the percent area of the tissue section that stained for Tgm2. Tgm2 was significantly elevated in post mortem COVID tissue compared to health control tissue *=P<0.05 by ANOVA.

### EXAMPLES

### Material

Anti-TG2 antibody: the anti-TG2 mAb that was used in the following examples comprised a light chain variable region as defined in SEQ ID NO: 25 and a heavy chain variable region as defined in SEQ ID NO: 38. It is a rabbitised version of the original BB7, and is herein named rbBB7 in the following examples.

Zampilimab (also known as UCB7858; derived from the antibody DC1), an anti-TG2 antibody having a variable light chain according to SEQ ID No. 24 and a variable heavy chain according to SEQ ID NO: 37, is a humanised antibody binding specifically human TG2. In order to be able to mimic its effects on animal models, such as rabbit, rbBB7 has been developed. Zampilimab/DC1 and rbBB7/BB7 have been shown to behave in a similar way. They bind to the same epitope in the TG2 core (aa 313-325 of SEQ ID No. 41), have almost identical IC50 (0.25 vs 0.3 nM) and Kd (<50 vs<60 pm) against human TG2) and inhibit ECM accumulation comparably in in vitro cell based assays. The only notable difference is the inferior IC50 of Zampilimab against rabbit TG2 (103 vs 8 nM). Therefore, the findings from the following examples using rbBB7 are fully applicable to Zampilimab and any other of the anti-TG2 antibodies, such as the ones herein described.

### Example 1—Collagen and TG2 Expression in IPF

The objective of this study was to determine if the presence of collagen and TG2 expression were associated with pulmonary fibrosis in human patients.

### Methods

Human tissues: Lung biopsy samples were obtained from the NIH and University of Rochester. Samples from 10 non-fibrotic and 10 IPF patients were analysed.

Preparation of the tissue samples: Formalin fixed, paraffin embedded human samples were serial sectioned and stained to correlate extent of fibrosis to TG2. Staining was performed to assess for fibrosis as measured by collagen content and transglutaminase type 2 (TG2) mRNA and protein expression.

Picrosirius Red Staining (PSR): Sample sections were stained with picrosirius red, which stains collagen fibrils bright red, while non-collagen areas are stained pink or yellow-brown. Whole slide scans were obtained using a Zeiss Axio Z.1 Scanner with Zeiss Zen 2.6 (Blue Edition) software. Images were processed using Definiens Tissue Studio software using a multiphase analysis. The image processing created a mask of the tissue to identify regions as lesion (active fibrosis), parenchyma (pre-fibrosis), white space, or airway collagen. Within each mask percent positive PSR staining was calculated as follows:

([% AREA of mask]×[% positive staining within mask])/100=% collagen in region

The sum of lesion and parenchyma % collagen in region represents the total percent non-airway collagen within the biopsy.

TG2 protein expression: Immunohistochemistry for TG2 was performed using the automated staining platform Leica Bond RX using a staining protocol of Dewax (Bond Dewax Solution at 72° C. 30 mins), H1 (20) antigen retrieval (100° C. with Bond ER solution 1) and DAB 30 Min Marker detection (using mouse anti TG2 antibody DH2 (UCB, internal antibody) at 83 ng/ml). Staining was quantified using a multiphase area analysis on Definiens tissue studio software.

TG2 mRNA: TG2 mRNA was assessed using the RNAscope In Situ Hybridization (ISH) assay in formalin-fixed, paraffin-embedded (FFPE) tissues performed on a Leica Bond RX processor with RNAscope 2.5 LS Reagent Kit Red (Advanced Cell Diagnostics) and Leica Bond Polymer Refine Red Detection Kit according to the manufacturer's instructions. Tissue quality was assessed by performing RNAscope analysis for mRNA of the housekeeping gene Homo sapiens ubiquitin C mRNA. Sections were taken at 5 μm thickness onto Superfrost Plus Gold slides and allowed to dry overnight at 37° C. followed by Leica Bond RX factory “Bake and Dewax” protocol.

The slides were placed on the staining rack of the Leica BOND RX without any pre-treatment and baked in position at 60° C. and then dewaxed before being rehydrated using ethanol. Heat-induced RNA retrieval was conducted by incubation in retrieval buffer ER2 (pH9, AR9640 Leica) for 15 min at 95° C., followed by protease treatment (Advanced Cell Diagnostics) for 15 min and peroxidase blocking with two rinses in distilled water between pre-treatments. Briefly, 20 ZZ probe pairs targeting the relevant genomic nucleoprotein genes were designed (target nucleotides Target 160-2563) and synthesized by Advanced Cell Diagnostics. Sections were exposed to ISH target probes and incubated at 42° C. for 2 hr. Hs-TGM2 (Advanced Cell Diagnostics). A probe to the bacterial gene DapB mRNA was used as a negative control for each run. After rinsing, the ISH signal was amplified using company-provided Pre-amplifier and Amplifier conjugated to alkaline phosphatase (AP) and incubated with a red substrate-chromogen solution for 10 min at room temperature. Sections were then counterstained with hematoxylin, air-dried, before mounting in Ecomount permanent mounting medium (Biocare Medical). Images were acquired on an Olympus slide scanner and quantified using Halo image analysis software by imaging specialist company Oracle Bio based on the number of cells staining positive for TG2 mRNA and the intensity of staining (number of probes) within each cell.

### Results

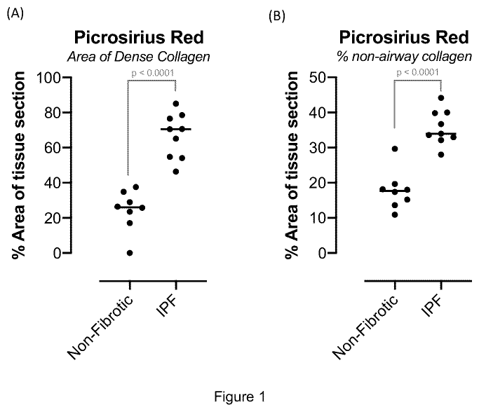

Presence of collagen (FIG. 1); Picrosirius red (PSR) staining was used to visualize collagen. Whole biopsy scans demonstrated more dense tissue and less air space in the IPF biopsies compared to the non-fibrotic samples, however some of these “non-fibrotic” controls clearly had some remodelling occurring (photographs not shown). Four regions were defined within the tissue: (1) Active fibrotic lesion area was assigned based on a high density of red collagen fibrils; (2) total parenchyma was defined by low density of red collagen fibrils in areas dominated by pink or yellow staining of non-collagen containing tissue; (3) airspace was defined by white spaced or unstained areas of the slide within the tissue periphery; and (4) airway collagen was defined as regions of dense collagen surrounding airways (this is a normal feature of lung histopathology and is not expected to change much with fibrotic disease).

Comparison of IPF and non-IPF sections demonstrated an increase in lesion size and high-density collagen area, coupled with a reduction in the air space and parenchyma area. There was also significant increase in percent non-airway collagen within the tissue (FIG. 1A/B).

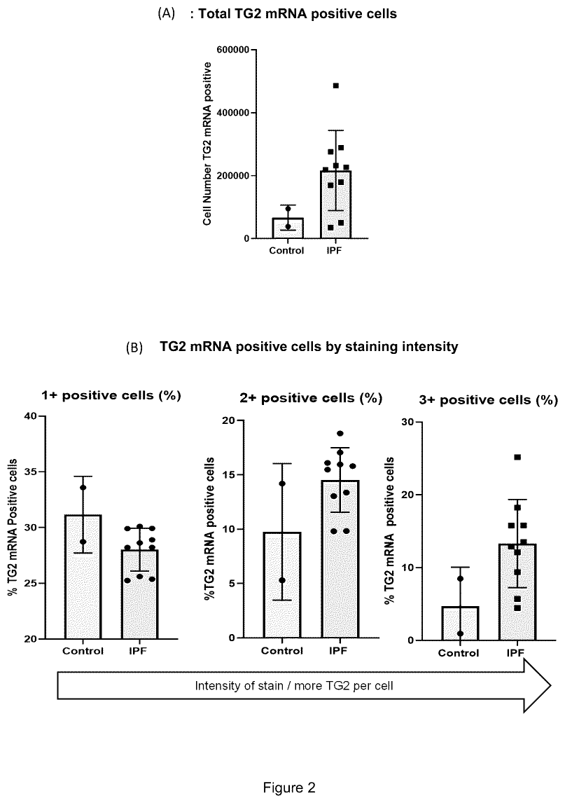

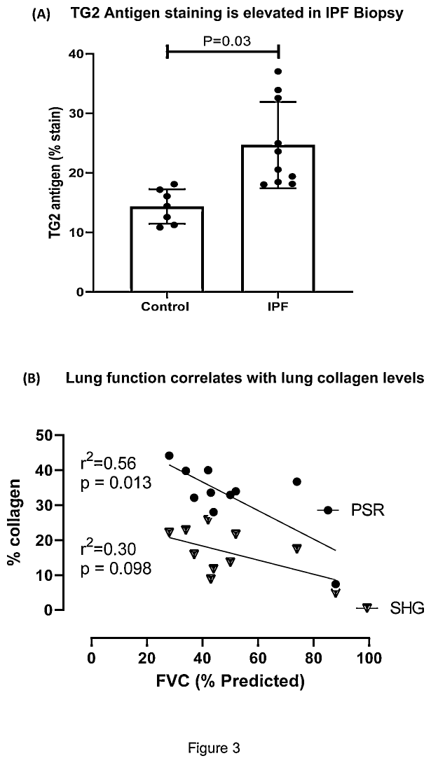

TG2 expression (FIGS. 2 and 3) Tissue transglutaminase 2 is encoded by the gene Tgm2. Quantification of the corresponding mRNA indicated a significant increase overall in the number of cells positive for TG2 mRNA per area in the IPF biopsies (FIG. 2A). The number of probes per cell was also quantified (FIG. 2B) by subdividing cells expressing low, medium and high TG2 RNA per cell. There was a significant 3-fold increase in the number of high TG2-mRNA expressing cells in the IPF group compared to non-fibrotic.

Immunohistochemistry staining for TG2 identified the localization of protein expression within the tissue. The observations showed TG2 positive staining localized to the epithelium in non-fibrotic tissue and stronger staining localized to fibrotic areas in IPF samples. A 42% increase in TG2 positive stained area was demonstrated within the whole biopsy in the IPF samples compared to non-fibrotic (FIG. 3A). As shown in FIG. 3B, decreased Forced vital capacity (FVC) was significantly associated with the percent of non-airway collagen as determined by PSR staining (circles; P=0.0128, R2=0.5597). Decreased FVC was also associated with collagen detected by SHG, although this was not significant (triangles, P=0.0983, R²=0.3043).

### Conclusion of Example 1

Total lung collagen was significantly increased in IPF tissues and was correlated (R² 0.56, p=0.01) with decreased lung function, consistent with the expected disease phenotype and prior reports. TG2 mRNA expression and TG2 protein detectable in fixed samples were increased in IPF lung tissues.

### Example 2—Knock-Out Animal Models

It was previously shown that TG2-knock out mice were protected from fibrotic remodelling in the lung bleomycin model of interstitial lung disease (Olsen et al., 2011). However, there remains an absolute need to link histology protection to function benefit in the lung when TG2 is reduced.

### Methods

All animal procedures were carried out under approved accreditations.

Wildtype & TG2 knock-out mice: Mice used for these experiments were male or female C57BL/6J purchased commercially (The Jackson Laboratories, n=20) or TG2 knockout mice bred in-house (n=21). The TG2 knockout mice were strain Tgm2tm1.1Rmgr (Victor Chang Institute) in which exons 6-8 of the Tgm2 gene were removed by Cre-mediated recombination. The strain was subsequently backcrossed at least 10 generation to C56BL/6J, which was verified by genome scanning.

Bleomycin model: Mice received either 2U/kg bleomycin (Fresenius Kabi) in 40 μl saline or just 40 μl saline by oropharyngeal aspiration while under isoflurane anaesthesia. After 21 days, the mice were euthanized according to a method consistent with the most recent guidelines from the Panel on Euthanasia of the American Veterinary Medical Association. The heart and lungs were removed en bloc. The right bronchus was tied off, and the right lung lobes were snap frozen in liquid nitrogen. The left lung was inflated with 2% low melting point agarose and placed in 10% neutral buffered formalin or was inflated with neutral buffered formalin and then placed in formalin overnight.

Histology: Mouse lung pieces were fixed in 10% neutral buffered formalin overnight, then transferred to 70% ethanol until embedding in paraffin. 5-micron sections were stained with H&E, Masson's Trichrome, or Picrosirius Red, by standard methods. For some experiments, collagen staining was quantified with Oracle Definiens Tissue studio software.

Lung function testing: For lung function testing, the mice were anesthetized, the tracheas were cannulated, and the mice were connected to a ventilator attached to a FlexiVent instrument (SciReq). The mice were paralyzed with 2 mg/kg vecuronium bromide i.p. to prevent spontaneous breathing effort, and the Flexivent performed a series of forced ventilation manoeuvres that were used to derive lung function parameters, usually lasting about 10 minutes. The mice were removed from the ventilator, euthanized, and tissue harvested.

RNA quantification: The right middle lung lobe was flash frozen in liquid nitrogen at time of euthanasia and stored at −80° C. until RNA purification. RNA was purified using Qiazol extraction with tissue pulverization with a bullet blender, followed by purification with RNAeasy kit (Qiagen,) according to manufacturer protocols. Reverse transcription to cDNA was completed with iScript Supermix (Bio-Rad). Real-time PCR was completed with 1 ng of sample cDNA, and pre-validated rabbit primers targeting COL1A1, COL3A1, fibronectin and Tgm2, as well as GADPH (housekeeping gene).

### Results:

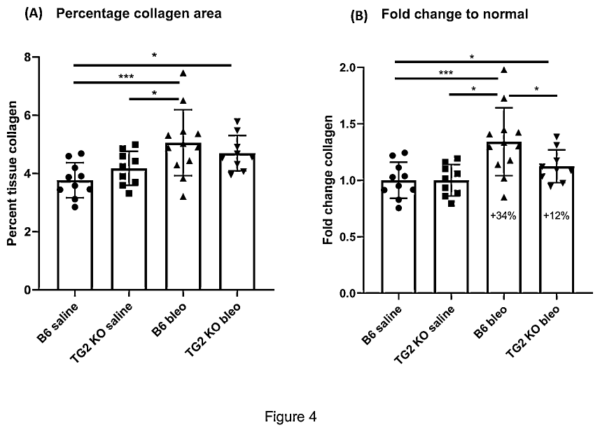

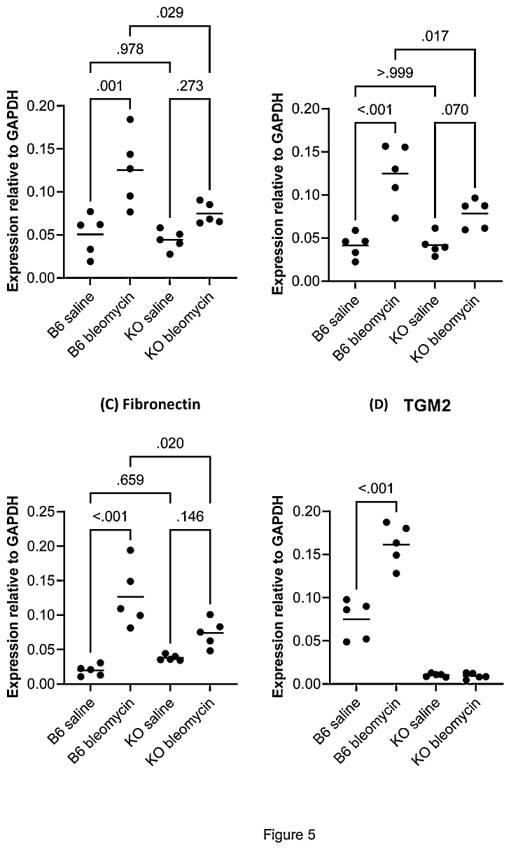

Lung collagen expression (FIGS. 4 and 5): Second harmonic generation assessment of lung collagen (FIG. 4) confirmed the previous cytochemical staining assessment of a reduced increase in collagen levels in the TG2 KO mice post bleomycin treatment compared with wild type C56BL/6 mice (Olson et al., 2011). However, TG2 deficiency resulted in a definite and significant reduction in interstitial collagen gene expression that was greater than could have been realistically expected given current understanding of TG2 mechanism of action in fibrosis (FIG. 5).

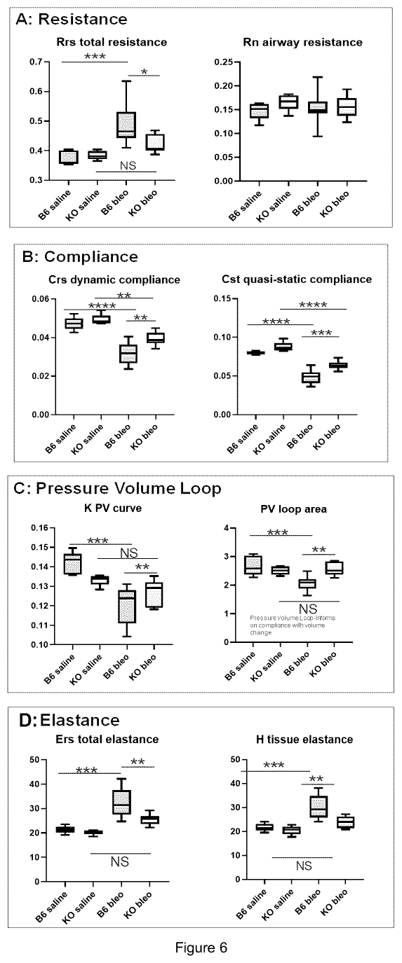

Lung function after bleomycin treatment (FIG. 6): Prior to tissue harvest, pulmonary function was measured in the mice with a FlexiVent apparatus. Two measures of resistance in the respiratory system to forced ventilation were made. The first, Rrs, or total resistance, is the resistance of the whole respiratory system to forced ventilation. Rrs was significantly increased in C57BL/6 mice in response to bleomycin but the small increase in TG2 KO mice was not significant, and significantly lower than the Rrs in the C57BL/6 mice (FIG. 6A). The second, Rn, or Newtonian resistance, is an estimate of how much of the total system resistance is due to changes in airway resistance (such as with airway narrowing in asthma). In both wildtype and TG2 KO mice Newtonian resistance was not affected by bleomycin (FIG. 6A). On assumption that the chest wall was also not affected by bleomycin, the clear differences between wildtype and KO mice in Rrs must be due to changes in the resistance of the parenchymal lung tissue (i.e. interstitial lung disease).

Compliance describes the ease with which the respiratory system can be extended. Compliance typically decreases in fibrosis because the lungs are stiffer, scarred and less able to expand. Dynamic compliance is measured during tidal breathing. Static compliance is measured in human patients during a deep breath-holding manoeuvre—as mice can't hold their breath on command, the Flexivent instrument measures quasi-static compliance during a single deep inflation by the ventilator (as mice don't hold their breath on command). Bleomycin caused a significant loss of both dynamic and quasi-static compliance in the C57BL/6 mice (FIG. 6B). TG2 KO mice also experienced decreased compliance, but this was preserved relative to C57BL/6 and they only lost about half the compliance of C57BL/6 mice.

PV loop area reflects the ability of a forced ventilation manoeuvre to recruit alveoli to breathing; a smaller PV loop area indicates less recruitable alveolar volume. K measures the curvature of the deflation limb of the PV loop; decreased K suggests more rapid initial deflation after a breath, which would be consistent with increased elastic stiffness (the lungs don't stretch as much on inflation so they collapse faster on deflation). The C57BL/6 mice had a significant drop in both the K PV curve and PV loop area in response to bleomycin (FIG. 6C). In contrast the TG2 KO mouse was protected from this drop with no significant decrease in either.

Elastance measures the elastic stiffness of the lung tissue and is the reciprocal of compliance. Ers is the elastance of the total respiratory system and includes contributions from the tissue, the chest wall and the airways, while H is the elastance of the tissue only, calculated from two different mathematical models of lung tissue. Relative to saline-treated mice, bleomycin significantly increased the elastic stiffness of the lung tissue in the C57BL/6 mice but not TG2 KO mice (FIG. 6D). This change in total system elastance (Ers) is attributed to changes in the lung tissue, in line with the finding related to the elastance of the tissue (H).

Taken together, TG2 KO mice were functionally protected in the bleomycin model of interstitial lung disease across all parameters used to assess respiratory function. They had no increase in total resistance, no increase in elastic stiffness and no loss of pressure volume loop with a significantly reduced loss in compliance than wild type mice.

### Conclusion of Example 2:

It was a finding that TG2 KO mice were protected from loss of lung function after bleomycin induced interstitial lung fibrosis. The magnitude of the protection was particularly surprising. C57BL/6 mice exhibited decreased compliance, increased elastic stiffness, increased tissue resistance, and decreased airspace recruitment, with bleomycin, all of which are consistent with human IPF and other fibrosing ILDs. To the contrary, the TG2 KO mice were protected from these effects, and showed no change in airspace recruitment or resistance, and about 50% less decrease in compliance and increase in elastance. These surprising and exciting results demonstrate the protective effect of TG2 deficiency not only on tissue histology but also and most importantly lung function.

### Example 3—In Vitro Inhibiting Activities of an Anti-TG2 Antibody

### Methods

Scratch wound experiments: Primary lung fibroblasts were cultured in ATCC RPE medium supplemented with calcium for 7 days. Some cells were left untreated and some treated with 1 ng/ml TGFβ as indicated. The monolayer was scratched and 250UM of TG2 substrate 5-BP was added either immediately, after 1 hour or after 3 hours. The cells were then harvested, washed and fixed in methanol 1 hour after addition of 5-BP (whole cell mount). Fibronectin was detected by antibody staining and TG2 activity was detected with HRP-streptavidin to detect the 5-BP label. The intensity of the fluorescent signal was quantified at the scratch boundary and in the cell layer away from the scratch. At the short incubation time used, 5-BP is only detecting extracellular or cell surface TG2 activity and not intracellular activity.

### Results

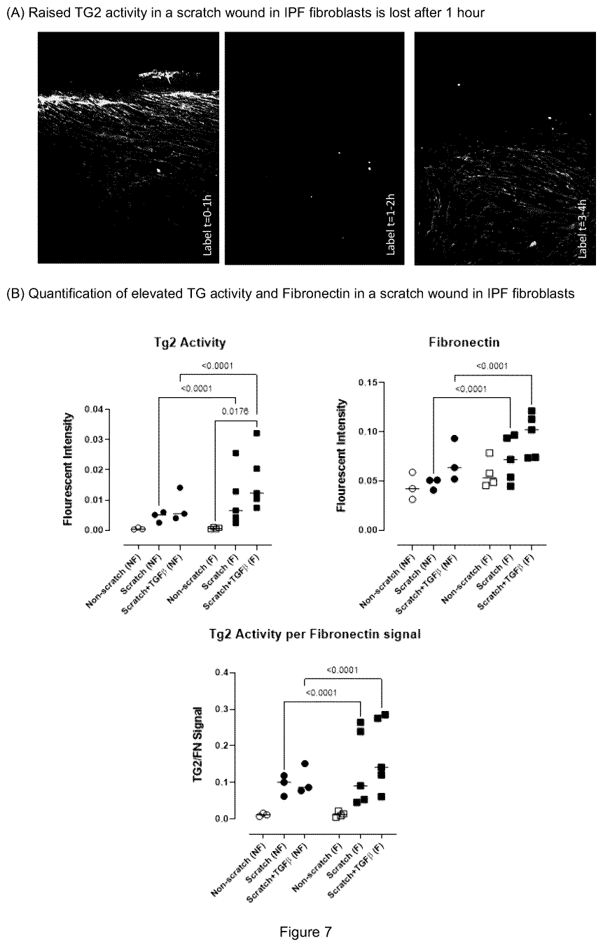

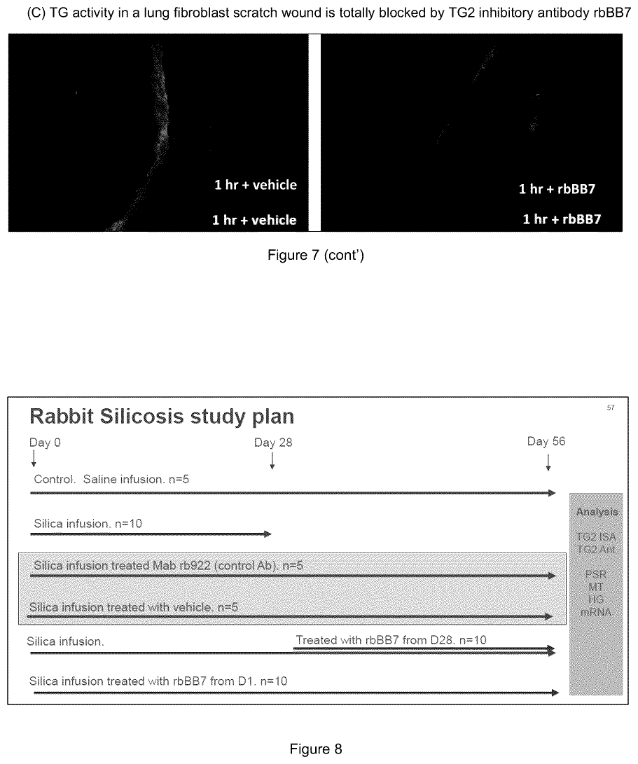

Quantitation and inhibition of TG2 activity in a scratch assay (FIG. 7); Scratch assays were performed on 8 cell primary cell lines from different human donors (3 non-fibrotic and 5 IPF) in both native and TGFβ1 treated states. TG2 activity was rapidly elevated post scratching as demonstrated by the incorporation of transglutaminase substrate 5-biotinylated pentylamine (5BP) into the cell extracellular matrix as visualised by raw fluorescent intensity (FIG. 7A left panel). However using timed administration of the substrate and stopping the study 1 hour post substrate administration of the 5BP, this activation was shown to be transient with all activity lost within 1 hour of the scratch, with no activity recorded when 5BP was applied 1 hour post scratch (FIG. 7A middle and right panels). As expected, IPF cell strains expressed more fibronectin than non-fibrotic strains. Both fibrotic and non-fibrotic strains showed increased TG2 activity after a scratch, and IPF strains had significantly more activity than non-fibrotic strains. Activation of TG2 is potentiated by TGFβ and is greater in IPF fibroblasts than non-fibrotic fibroblasts (FIG. 7B). This is an important finding with implications for the role of TG2 in IPF. Administration of a TG2 inhibitory antibody (rbBB7) was able to completely block the elevated TG activity along the scratch confirming that the increase in activity is due solely to TG2 and the TG2 is elevated extracellularly in response to wounding.

### Conclusions of Example 3:

Primary human lung fibroblasts in culture export TG2 into the extracellular space, but it is inactive unless activated by a scratch wound. Activation is transient, lasting less than 1 hour. Fibroblasts from donors with IPF export more TG2 activity than non-fibrotic fibroblasts. This activity can be completely inhibited by rbBB7. This data provides evidence that activated TG2 in IPF can be inhibited with anti-TG2 antibodies such as rbBB7 or Zampilimab. This data constitutes an anti-fibrotic response for Zampilimab in primary human lung cells.

### Example 4—In Vivo Inhibiting Activities of an Anti-TG2 Antibody

### Methods

Interventional studies with rbBB7, vehicle or control antibody treatments: The overall study design (FIG. 8) used 45 animals (rabbits) using instillation of 10 mg of silica with preventative and therapeutic treatment arms running over 56 days.
5 rabbits received saline and were harvested at day 56 (healthy group).
10 rabbits received silica and were harvested at day 28 (level of disease on starting therapeutic treatment)
10 rabbits received silica and began treatment with rbBB7 (100 mg/kg) at day 28 with harvest at day 56 (therapeutic dosing)
10 rabbits began treatment with rbBB7 (100 mg/kg) 1 day before instillation of silica (i.e. treatment at day-1) and were harvested at day 56 (prophylactic dosing).
5 rabbits received silica and began treatment with rb922 control antibody (100 mg/kg) 1 day before instillation of silica and were harvested day 56 (control antibody).
5 rabbits received silica and began treatment with vehicle one day before instillation of silica and were harvested at day 56 (vehicle control)

The design would allow for combination of antibody controls and vehicle controls to form an untreated group of 10 animals if there were no differences between these groups

Rabbits were injected with rbBB7, rb922 control antibody or vehicle every 5 days, subcutaneously in the scruff of the neck. The rabbits were weighed prior to injection and the antibody volumes injected adjusted accordingly to give a 100 mg/kg dose. Rabbits receiving the control vehicle were given the same volume of vehicle that they would have received based on their weight, if they had been receiving antibody. The average starting weight of the rabbits was 2.5 kg and the average weight at the end of 56 days was 3.2 kg.

Pharmacokinetics: Every 5 or 10 days, blood was drawn from the rabbits being treated with rbBB7. The first blood draw was 5 days after the first rbBB7 injection. Each rabbit was sampled a maximum of four times to minimize risks for the animal, Blood draws were always performed immediately prior to the next scheduled injection of rbBB7 to calculate trough (minimum) exposure values. Prophylactic (from day-1, plot A and B) and therapeutic (day 26 to 56, plot C and D) dosing were both performed in 2 separate runs and thus plotted individually. The blood was allowed to clot at room temperature for 45 minutes, then was centrifuged at 300×g for 15 minutes. Serum was decanted and stored frozen at −80° C. rbBB7 was quantified in the serum using Mass Spectrometry.

Tissue Processing: The lungs were inflated with 30% sucrose in PBS through the trachea, as a cryoprotectant, and to give them some resistance to the cutting blades so they did not tear or crush as easily. The lower right lobe was placed in a custom block and sliced into 5-6 pieces each about 4 mm thick. The central piece was placed in 10% formalin for 24 hours, then transferred to 70% ethanol, and processed for histology. The left and right (central and distal) slices were frozen at −80° C. for later analysis.

Histology and Analysis: The formalin-fixed lung slice was processed as a single piece and embedded in a single large paraffin block. Sections were cut and stained with H&E, Trichrome and Picrosirius red (PSR), and imaged on either an Olympus or Zeiss slide scanner. For quantitation of collagen, the PSR whole slide images were processed using a multiphase analysis using Definiens Tissue studio software. The software ran an algorithm to identify “lesion” and non-lesion/unaffected parenchyma) areas based on cellular density and loss of unstained airspaces. Then, collagen (bright red stained fibres) in both lesion and non-lesion areas were quantified as a percentage of the total region area. Analysis of the same paraffin sections was repeated using multiphoton microscopy/second harmonic generation (SHG) to quantify collagen using a Genesis 200 scanner. SHG microscopy is a variant of two photon (2P) microscopy that can detect the fibrillar collagens without exogenous labels. The fibril-forming collagens include collagen types 1-3, 5, 11, 24, and 27. Several of these fibrillar collagens such as types I, III, and V are key players in lung fibroses (Kottmann et al. 2015).

TG2 Antigen and Activity Immunohistochemistry: TG2 antigen was detected by immunostaining on frozen slices of lung tissue (that had been inflated with 30% sucrose at harvest and then snap-frozen), according to standard protocols. Transamidase (crosslinking) activity was measured on frozen slices of lung tissue using a Transglutaminase in situ activity (TG ISA) assay using the incorporation of biotinylated cadaverine as previously described. The use of sucrose to inflate lungs interferes with this assay, and the extra wash cycles needed to remove the sucrose lowered measured TG activity considerably. Even though absolute TG2 activity is lower than in similar experiments without sucrose, the relative differences between groups should be the same, since all the tissues were treated the same.

### Results

Interventional study with rbBB7: 43 of 45 animals successfully completed the experimental regimen (two rabbits died during the initial silica instillation, likely due to anaesthesia effects). There were no detrimental effects observed in rbBB7 treated rabbits. They showed similar weight gain to control rabbits (data not shown) and did not develop any physical issues related to repeated injection of the therapeutic antibody.

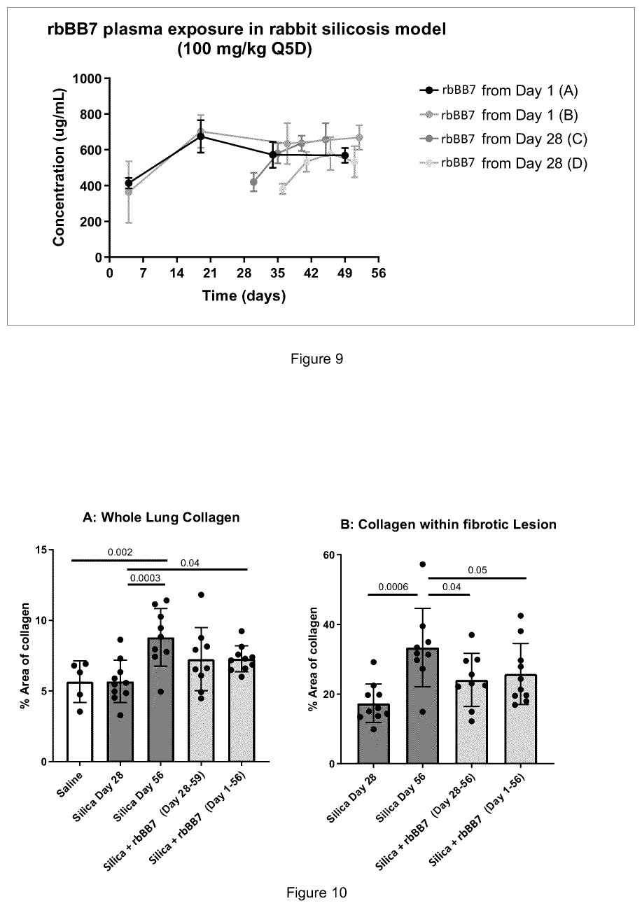

Pharmacokinetics (FIG. 9): 5 days after the first rbBB7 injection (Day 4 post silica, the first sample for trough PK), serum levels averaged 395 μg/ml. By the next sampling point (Day 19) rbBB7 levels were in the region of 700 μg/ml and maintained a steady state average of 600-700 μg/ml (when measured 5 days after the previous injection at trough). There was no significant decline in the antibody levels over the 56 days which might have indicated the development of an allergic or anti-antibody response. These levels are expected to be therapeutic in the rabbit model. Sampling did not occur after every treatment in order to reduce stress to the animals.

Histological Analysis of Fibrosis (FIG. 10); Rabbit lung sections stained with H&E or Picrosirius red demonstrate the presence of fibrosis in the silica treated rabbits (pictures not shown). To quantify the extent of fibrosis, Definiens image analysis was performed on the PSR stained sections. The software quantified collagen (red) staining both within just the lesion areas and the lung lobe as a whole (i.e. normal parenchyma or non-lesion areas plus lesion area) (pictures not shown). Looking at whole lobe staining there was no clear change in PSR staining by 28 days but increased on average by 35% between days 28 and 56 (FIG. 10A). A significant treatment effect of rbBB7 in reducing the amount of collagen was seen. If looking at total lobe staining there was a 50% reduction in the increase of collagen in both the protective and therapeutic dosing groups, although this only reached significance in the protective regimen. Assessment of collagen PSR staining with just lesions also showed a marked reduction in the 92% increase in collagen between days 28 and 56, with significant 58% and 48% reductions in therapeutic and protective arms respectively (FIG. 10B).

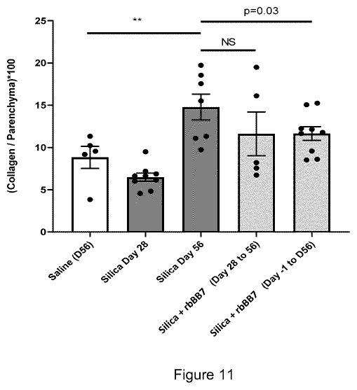

Analysis of collagen content by Second harmonic generation (SHG) microscopy (FIG. 11); SHG was used to detect total fibrillar collagens in the silica treated lobe. Rabbit lung tissue sections were scanned, and the collagen was quantified. SHG detected no increase in collagen at day 28 post silica instillation and may reflect that the collagen may not be mature enough to detect by SHG. However, the level of detectable fibrillar collagen more than doubled between day 28 and day 56 post silica instillation. Treatment with rbBB7 reduced collagen accumulation in both the early and late treatment groups markedly. Using the mean level of SHG detectable collagen as a baseline, there was a 52% reduction in both treatment arm, although only the protective treatment group reached significance.

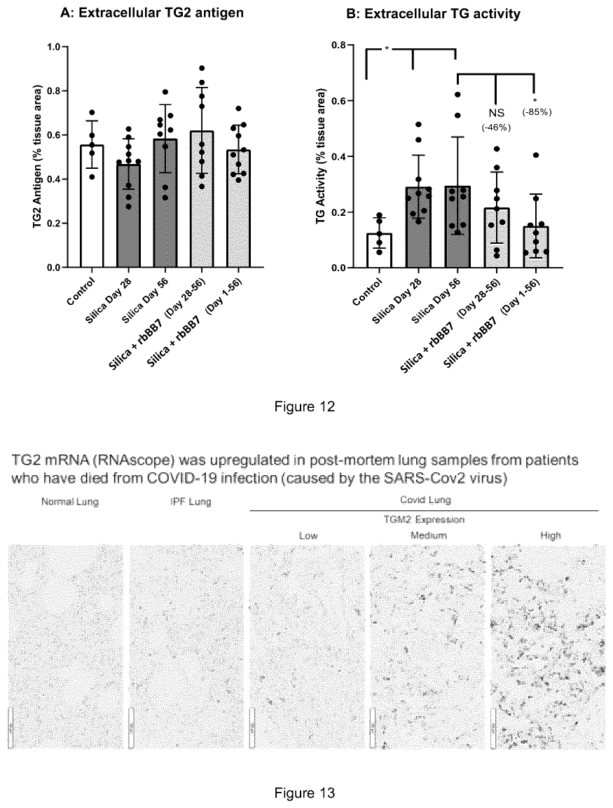

Changes in TG2 activity (FIG. 12); Frozen sections were prepared from one of the frozen lung slices, and TG2 antigen and activity were measured. TG2 antigen levels were unchanged by instillation of silica and not affected by rbBB7 treatment (FIG. 12A). Despite the use of sucrose in the fluid used to inflate the lungs which significantly compromises the TG ISA assay, TG activity was significantly increased 2.3-fold with silica treatment both at day 28 and day 56 post silica instillation. RbBB7 reduced TG2 activity in both silica groups (FIG. 12B). The 45% reduction in the therapeutic dosing arm did not reach significance (likely due to high level of variability), but the 85% reduction in the protective arm was significant and comparable to levels in the normal lung.

### Conclusion of Example 4

It is a surprising finding of this example that 1) both early preventative treatment (therapy given every 5 days starting the day before silica instillation) and late treatment (therapy given every 5 days starting 28 days after silica instillation) were effective in reducing histological fibrosis, and 2) as an antibody inhibitor, only able to target extracellular TG2, was able to protect the animals from fibrosis as well as total TG2 knockout (in the mouse model), indicating lung fibrosis is mainly due to extracellular TG2 activity. The reduction in fibrosis was independently confirmed by two methods of analysing collagen in lung tissue (PSR staining and SHG microscopy). The anti-TG2 antibody also blocked TG2 enzymatic activity as detected in frozen lung sections. In other words, an anti-TG2 antibody (that inhibits extracellular protein crosslinking activity of TG2) was able to significantly attenuate pulmonary fibrosis in a rabbit model of silicosis when given preventively, and arrest further progression of fibrosis when given starting 28 days after initiation of lung remodelling.

### Example 5—TG2 is Upregulated in COVID-19 Infection

### Methods

Human tissues: Lung tissue samples were obtained from Tissue Solutions Ltd. Samples from 22 patients who had died from COVID-19 infection, and 6 tissue samples of normal lung and IPF patients were analysed. Patients having known lung disease, including chronic obstructive pulmonary disease (COPD) were excluded.

Preparation of the tissue's samples: Formalin fixed, paraffin embedded human samples were serial sectioned and staining was performed to assess transglutaminase type 2 (TG2) mRNA expression.

TG2 mRNA (RNAscope): A similar protocol as the one detailed in Example 1 was used.

### Results

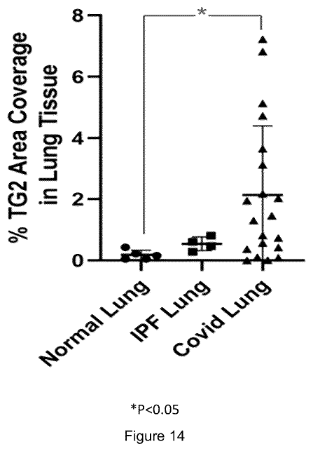

As shown in FIG. 13 and FIG. 14, TG2 mRNA expression was significantly upregulated (P<0.05) in post-mortem tissue samples obtained from patients who had died with COVID-19 compared to expression in normal lung tissue. IPF samples were included for reference only. This high expression is accompanied by evidence of extensive matrix deposition (picrosirus red staining—data not shown).

### Conclusion of Example 5

It was shown by the inventors that during the inflammatory response and remodeling of lung tissue occurring with severe SARS-cov2 viral infection that TG2 is very highly expressed in the majority of post-mortem lung samples from patients who have died from COVID-19 infection. It is anticipated that anti-TG2 antibodies, such as Zampilimab could slow the remodeling not only during the acute phase but also at long term chronic phase of COVID disease.

### REFERENCES

1) Siegel et al., 2007, Pharmacol. Ther., 115 (2): 232-245
2) Wang et al., 2020, 3 Biotech., 10:287
3) WO2006100679,)
4) WO2012146901
5) WO2013175229
6) Olsen et al., 2011, Am. J. Respir. Crit. Care Med., 184:699-707
7) Olsen 2020, Am. J. Respir. Crit. Care Med., 201: A1963
8) Philp et al., 2018, Am. J. Resp. Cell Mol. Biol., 58 (5): 594-603
9) Olsen et al., 2014, Am. J. Resp. Cell Mol. Biol., 50 (4): 737-747
10) Raghu, 2017, Eur. Respir. Rev. 2017; 26:170071
11) Vaidya et al., 2017, Curr. Med. Chem., 24:1-20
12) Margaritopoulos et al., 2016, Core Evidence, 11:11-22
13) WO2015/197772
14) Kottmann et al., 2015, Resp. Res., 16:61

1. An anti-Transglutaminase 2 (anti-TG2) antibody for use in the treatment of a subject having progressive chronic interstitial lung disease or in the prevention of the development of progressive chronic interstitial lung disease.

2. The anti-TG2 antibody for use according to claim 1, wherein the lung disease is selected from the group consisting of Idiopathic pulmonary fibrosis (IPF), Desquamative interstitial pneumonia (DIP), Acute interstitial pneumonia (AIP; alternatively known as Hamman-Rich syndrome), Hypersensitivity pneumonitis (HSP), Nonspecific interstitial pneumonia (NSIP), Respiratory bronchiolitis-associated interstitial lung disease (RB-ILD), Cryptogenic organizing pneumonia (COP; alternatively named Bronchiolitis Obliterans Organizing Pneumonia or BOOP), sarcoidosis, asbestosis and Lymphoid interstitial pneumonia (LIP).

3. An anti-Transglutaminase 2 (anti-TG2) antibody for use in the treatment of a subject having a lung fibrosis associated with COVID infection or in the prevention of the development of a lung fibrosis in a subject suffering from COVID infection.

4. The anti-TG2 antibody for use according to claim 1, wherein the lung disease or the lung fibrosis is characterised by an increase of a marker in a subject's sample, said marker being selected from the group of TG2 activity, mRNA encoding TG2, TG2 antigen, enhanced export of TG2 or any combination thereof.

5. The anti-TG2 antibody for use according to claim 1, wherein said antibody binds to an epitope within the core region of transglutaminase type 2 (TG2) and inhibits TG2 activity, wherein said core region consists of amino acids 143 to 473 of TG2, and wherein the TG2 activity that is inhibited is the TG2 cross-linking of lysine and glutamine with N-ε(γ-glutamyl) lysine isopeptide bonds.

6. The anti-TG2 antibody for use according to claim 1, wherein the antibody or antigen-binding fragment thereof:
a. comprises or consists of an intact antibody, or
b. comprises or consists of an antigen-binding fragment.

7. The anti-TG2 antibody for use according to claim 1, wherein said antibody comprises the following sequences:
(i)
(LCDR1; SEQ ID NO. 1)
KASQDINSYLT;
(LCDR2; SEQ ID NO. 2)
LVNRLVD;
(LCDR3; SEQ ID NO. 3)
LQYDDFPYT;
(HCDR1; SEQ ID NO. 4)
THAMS;
(HCDR2; SEQ ID NO. 5)
TISSGGRSTYYPDSVKG;
and
(HCDR3; SEQ ID NO. 6)
LISTY;
or
(ii)
(LCDR1; SEQ ID NO. 1)
KASQDINSYLT;
(LCDR2; SEQ ID NO. 7)
LTNRLMD;
(LCDR3; SEQ ID NO. 8)
LQYVDFPYT;
(HCDR1; SEQ ID NO. 9)
SSAMS;
(HCDR2; SEQ ID NO. 5)
TISSGGRSTYYPDSVKG;
(HCDR3; SEQ ID NO. 10)
and
LISPY;
(iii)
(LCDR1; SEQ ID NO. 1)
ASQDINSYLT;
(LCDR2; SEQ ID NO. 11)
RTNRLFD;
(LCDR3; SEQ ID NO. 3)
LQYDDFPYT;
(HCDR1)
SSAMS;
(HCDR2; SEQ ID NO. 9)
TISVGGGKTYYPDSVKG;
and
(HCDR3; SEQ ID NO. 12)
LISLY.

8. The anti-TG2 antibody for use according to claim 1, wherein the antibody comprises:
a) a light chain variable domain having the sequence as defined in any one of SEQ ID NO: 13 to SEQ ID No. 27 and a heavy chain variable domain having the sequence as defined in any one of SEQ ID NO: 28 to SEQ ID No. 40,
b) a light chain variable domain having at least 80% identity or similarity, preferably at least 90% identity or similarity, or preferably at least 95% identity or similarity to the sequence as defined in any one of SEQ ID NO: 13 to SEQ ID No. 27 and a heavy chain variable domain having at least 80% identity or similarity, preferably at least 90% identity or similarity, or preferably at least 95% identity or similarity to the sequence as defined in any one of SEQ ID NO: 28 to SEQ ID No. 40.

9. The anti-TG2 antibody for use according to claim 1, wherein said antibody competes for binding to TG2 with an antibody comprising the following sequences.
(i)
(LCDR1; SEQ ID NO. 1)
KASQDINSYLT;
(LCDR2; SEQ ID NO. 2)
LVNRLVD;
(LCDR3; SEQ ID NO. 3)
LQYDDFPYT;
(HCDR1; SEQ ID NO. 4)
THAMS;
(HCDR2; SEQ ID NO. 5)
TISSGGRSTYYPDSVKG;
and
(HCDR3; SEQ ID NO. 6)
LISTY;
or
(ii)
(LCDR1; SEQ ID NO. 1)
KASQDINSYLT;
(LCDR2; SEQ ID NO. 7)
LTNRLMD;
(LCDR3; SEQ ID NO. 8)
LQYVDFPYT;
(HCDR1; SEQ ID NO. 9)
SSAMS;
(HCDR2; SEQ ID NO. 5)
TISSGGRSTYYPDSVKG;
(HCDR3; SEQ ID NO. 10)
and
LISPY;
(iii)
(LCDR1; SEQ ID NO. 1)
ASQDINSYLT;
(LCDR2; SEQ ID NO. 11)
RTNRLFD;
(LCDR3; SEQ ID NO. 3)
LQYDDFPYT;
(HCDR1)
SSAMS;
(HCDR2; SEQ ID NO. 9)
TISVGGGKTYYPDSVKG;
and
(HCDR3; SEQ ID NO. 12)
LISLY.

10. A method for treating a subject having a progressive chronic interstitial lung disease or for the prevention of the development of a progressive chronic interstitial lung disease in a subject comprising administering a therapeutically effective amount of an anti-TG2 antibody to said subject.

11. A method for treating a subject having a lung fibrosis associated with COVID infection or for preventing the development a lung fibrosis in a subject suffering from COVID infection, wherein said method comprises the step of administering a therapeutically effective amount of an anti-TG2 antibody to said subject.

12. Use of an anti-Transglutaminase 2 (TG2) antibody for the manufacturing of a medicament for the treatment of a subject having a progressive chronic interstitial lung disease or for the prevention of the development of progressive chronic interstitial lung disease.

13. Use of an anti-Transglutaminase 2 (TG2) antibody for the manufacturing of a medicament for the treatment of a subject having a lung fibrosis associated with COVID infection or the prevention of the development a lung fibrosis in a subject suffering from COVID infection.


SEQUENCE LISTING (42 sequences)

SEQ ID NO: 1 - protein, 11 aa, synthetic construct
  REGION 1..11: note = L-CDR
KASQDINSYLT

SEQ ID NO: 2 - protein, 7 aa, synthetic construct
  REGION 1..7: note = L-CDR
LVNRLVD

SEQ ID NO: 3 - protein, 9 aa, synthetic construct
  REGION 1..9: note = L-CDR
LQYDDFPYT

SEQ ID NO: 4 - protein, 5 aa, synthetic construct
  REGION 1..5: note = H-CDR
THAMS

SEQ ID NO: 5 - protein, 17 aa, synthetic construct
  REGION 1..17: note = H-CDR
TISSGGRSTYYPDSVKG

SEQ ID NO: 6 - protein, 5 aa, synthetic construct
  REGION 1..5: note = H-CDR
LISTY

SEQ ID NO: 7 - protein, 7 aa, synthetic construct
  REGION 1..7: note = L-CDR
LTNRLMD

SEQ ID NO: 8 - protein, 9 aa, synthetic construct
  REGION 1..9: note = L-CDR
LQYVDFPYT

SEQ ID NO: 9 - protein, 5 aa, synthetic construct
  REGION 1..5: note = H-CDR
SSAMS

SEQ ID NO: 10 - protein, 5 aa, synthetic construct
  REGION 1..5: note = H-CDR
LISPY

SEQ ID NO: 11 - protein, 7 aa, synthetic construct
  REGION 1..7: note = L-CDR
RTNRLFD

SEQ ID NO: 12 - protein, 5 aa, synthetic construct
  REGION 1..5: note = H-CDR
LISLY

SEQ ID NO: 13 - protein, 107 aa, synthetic construct
  REGION 1..107: note = Light Chain
DIQMTQTPSSMYASLGERVTITCKASQDINSYLTWFQQKPGKSPKTLIYRTNRLFDGVPSRFSGSGSGQDFFLTISSLEYEDMGIYYCLQYDDFPYTFGGGTKLEIK

SEQ ID NO: 14 - protein, 107 aa, synthetic construct
  REGION 1..107: note = Light Chain of AB1 antibody
DIKMTQSPSSMYASLGERVTITCKASQDINSYLTWFQQKPGKSPKTLIYRTNRLFDGVPSRFSGSGSGQDFFLTISSLEYEDMGIYYCLQYDDFPYTFGGGTKLEIK

SEQ ID NO: 15 - protein, 107 aa, synthetic construct
  REGION 1..107: note = Light Chain
EIVLTQSPSSMYASLGERVTITCKASQDINSYLTWFQQKPGKSPKTLIYRTNRLFDGVPSRFSGSGSGQDFFLTISSLEYEDMGIYYCLQYDDFPYTFGGGTKLEIK

SEQ ID NO: 16 - protein, 107 aa, synthetic construct
  REGION 1..107: note = Light Chain
DIQMTQSPSSMYASLGERVTITCKASQDINSYLTWFQQKPGKSPKTLIYRTNRLFDGVPSRFSGSGSGQDFFLTISSLEYEDMGIYYCLQYDDFPYTFGGGTKLEIK

SEQ ID NO: 17 - protein, 107 aa, synthetic construct
  REGION 1..107: note = Light Chain
EIVLTQSPSSLSASVGDRVTITCKASQDINSYLTWYQQKPGKAPKLLIYRTNRLFDGVPSRFSGSGSGTDFFFTISSLQPEDFGTYYCLQYDDFPYTFGGGTKLEIK

SEQ ID NO: 18 - protein, 107 aa, synthetic construct
  REGION 1..107: note = Light Chain
DIQMTQSPSSLSASVGDRVTITCKASQDINSYLTWFQQKPGKAPKSLIYRTNRLFDGVPSRFSGSGSGTDFFLTISSLQPEDFATYYCLQYDDFPYTFGQGTKVEIK

SEQ ID NO: 19 - protein, 107 aa, synthetic construct
  REGION 1..107: note = Light Chain of BB7 antibody
AIKMTQSPSSMYASLGERVIITCKASQDINSYLTWFQQKPGKSPKTLIYLTNRLMDGVPSRFSGSGSGQEFLLTISGLEHEDMGIYYCLQYVDFPYTFGGGTKLEIK

SEQ ID NO: 20 - protein, 107 aa, synthetic construct
  REGION 1..107: note = Light Chain
DIQMTQSPSSLSASVGDRVTITCKASQDINSYLTWFQQKPGKAPKSLIYLTNRLMDGVPSRFSGSGSGTDFFLTISSLQPEDFATYYCLQYVDFPYTFGQGTKVEIK

SEQ ID NO: 21 - protein, 107 aa, synthetic construct
  REGION 1..107: note = Light Chain
DIKMTQSPSSLSASVGDRVTITCKASQDINSYLTWFQQKPGKAPKTLIYLTNRLMDGVPSRFSGSGSGQEFLLTISSLQPEDFATYYCLQYVDFPYTFGQGTKVEIK

SEQ ID NO: 22 - protein, 107 aa, synthetic construct
  REGION 1..107: note = Light Chain of DC1 antibody
DITMTQSPSSIYASLGERVTITCKASQDINSYLTWFQQKPGKSPKILIYLVNRLVDGVPSRFSGSGSGQDYALTISSLEYEDMGIYYCLQYDDFPYTFGGGTKLEIK

SEQ ID NO: 23 - protein, 107 aa, synthetic construct
  REGION 1..107: note = Light Chain
DIQMTQSPSSLSASVGDRVTITCKASQDINSYLTWFQQKPGKAPKSLIYLVNRLVDGVPSRFSGSGSGTDFFLTISSLQPEDFATYYCLQYDDFPYTFGQGTKVEIK

SEQ ID NO: 24 - protein, 107 aa, synthetic construct
  REGION 1..107: note = Light Chain of Zampilimab
DITMTQSPSSLSASVGDRVTITCKASQDINSYLTWFQQKPGKAPKILIYLVNRLVDGVPSRFSGSGSGQDYALTISSLQPEDFATYYCLQYDDFPYTFGQGTKVEIK

SEQ ID NO: 25 - protein, 107 aa, synthetic construct
  REGION 1..107: note = Light Chain of rbBB7 antibody
AIKMTQTPSSVSAAVGGTVTISCKASQDINSYLTWFQQKPGQPPKTLIYLTNRLMDGVPSRFSGSGSGQEFLLTISGLECDDAATYYCLQYVDFPYTFGGGTKVVVK

SEQ ID NO: 26 - protein, 112 aa, synthetic construct
  REGION 1..112: note = Light Chain of IA12 antibody
DVVNTQTPLTLSVTFGQPASISCKSSQSLLYDNGKTYLHWLFQRPGQSPRRLIYLVSKLDSGVPDRFTGSGSGTDFTLKISRVEAEDLGVYYCVQGTHFPYTFGGGTKLEIK

SEQ ID NO: 27 - protein, 106 aa, synthetic construct
  REGION 1..106: note = Light Chain
QIVLTQSPAIMSASPGEKVTMTCSASSSVSYMYWYQQKPGSSPRLLIYDTSNLASGVPVRFSGSGSGTSYSLTISRMEAEDAATFYCQQWSSSPLTFGAGTKLELK

SEQ ID NO: 28 - protein, 114 aa, synthetic construct
  REGION 1..114: note = Heavy Chain
EVQLEESGGGLVKPGGSLKLSCAASGFTLSSSAMSWVRQTPDRRLEWVATISVGGGKTYYPDSVKGRFTISRDNAKNTLYLQMNSLRSEDTAMYYCAKLISLYWGQGTTLTVSS

SEQ ID NO: 29 - protein, 114 aa, synthetic construct
  REGION 1..114: note = Heavy Chain of AB1 antibody
EVQLVESGGGLVKPGGSLKLSCAASGFTLSSSAMSWVRQTPDRRLEWVATISVGGGKTYYPDSVKGRFTISRDNAKNTLYLQMNSLRSEDTAMYYCAKLISLYWGQGTTLTVSS

SEQ ID NO: 30 - protein, 114 aa, synthetic construct
  REGION 1..114: note = Heavy Chain
EVQLQESGGGLVKPGGSLKLSCAASGFTLSSSAMSWVRQTPDRRLEWVATISVGGGKTYYPDSVKGRFTISRDNAKNTLYLQMNSLRSEDTAMYYCAKLISLYWGQGTTLTVSS

SEQ ID NO: 31 - protein, 114 aa, synthetic construct
  REGION 1..114: note = Heavy Chain
EVQLLESGGGLVQPGGSLRLSCAASGFTFSSSAMSWVRQAPGKGLEWVSTISVGGGKTYYPDSVKGRFTISRDNSKNTLYLQMNSLRAEDTAVYYCAKLISLYWGQGTLVTVSS

SEQ ID NO: 32 - protein, 114 aa, synthetic construct
  REGION 1..114: note = Heavy Chain of BB7 antibody
AVQLVESGGGLVKPGGSLKLSCAASGIIFSSSAMSWVRQTPEKRLEWVATISSGGRSTYYPDSVKGRFTVSRDSAKNTLYLQMDSLRSEDTAIYYCAKLISPYWGQGTTLTVSS

SEQ ID NO: 33 - protein, 114 aa, synthetic construct
  REGION 1..114: note = Heavy Chain
EVQLLESGGGLVQPGGSLRLSCAASGFTFSSSAMSWVRQAPGKGLEWVSTISSGGRSTYYPDSVKGRFTISRDNSKNTLYLQMNSLRAEDTAVYYCAKLISPYWGQGTLVTVSS

SEQ ID NO: 34 - protein, 114 aa, synthetic construct
  REGION 1..114: note = Heavy Chain
EVQLLESGGGLVQPGGSLRLSCAASGIIFSSSAMSWVRQAPGKGLEWVATISSGGRSTYYPDSVKGRFTVSRDSSKNTLYLQMNSLRAEDTAVYYCAKLISPYWGQGTLVTVSS

SEQ ID NO: 35 - protein, 114 aa, synthetic construct
  REGION 1..114: note = Heavy Chain of DC1 antibody
EVQLVESGGGLVKPGGSLKLSCAASGFTLSTHAMSWVRQTPEKRLEWVATISSGGRSTYYPDSVKGRFTISRDNVKNTLYLQLSSLRSEDTAVYFCARLISTYWGQGTTLTVSS

SEQ ID NO: 36 - protein, 114 aa, synthetic construct
  REGION 1..114: note = Heavy Chain
EVQLLESGGGLVQPGGSLRLSCAASGFTFSTHAMSWVRQAPGKGLEWVSTISSGGRSTYYPDSVKGRFTISRDNSKNTLYLQMNSLRAEDTAVYYCAKLISTYWGQGTLVTVSS

SEQ ID NO: 37 - protein, 114 aa, synthetic construct
  REGION 1..114: note = Heavy Chain of Zampilimab
EVQLLESGGGLVQPGGSLRLSCAASGFTLSTHAMSWVRQAPGKGLEWVATISSGGRSTYYPDSVKGRFTISRDNSKNTLYLQMNSLRAEDTAVYFCARLISTYWGQGTLVTVSS

SEQ ID NO: 38 - protein, 114 aa, synthetic construct
  REGION 1..114: note = Heavy Chain of rbBB7 antibody
EVQLLESGGRLVTPGGSLTLTCAASGIIFSSSAMSWVRQAPGKGLEWVATISSGGRSTYYPDSVKGRFTVSRDSSKNTLYLQMNSLRAEDTAVYYCAKLISPYWGQGTLVTVSS

SEQ ID NO: 39 - protein, 114 aa, synthetic construct
  REGION 1..114: note = Heavy Chain of IA12 antibody
QIQLVQSGPELKKPGETVKISCKASGYTFTTYGNTWVKQAPGKGLKWMGWINTSSGVPTYADDFKGRFAFSLETSASTAYLQINNLKSEDTATYFCARPEVAYWGQGTLVTVSA

SEQ ID NO: 40 - protein, 119 aa, synthetic construct
  REGION 1..119: note = Heavy Chain
QVTLKESGPGILQPSQTLSLTCSFSGFSLSTSGMGVSWIRQPSGKGLEWLAHIYWDDDKRYNPSLKSRLTISKDTSSNQVFLKITSVDTADTATYYCARSGTTAPFAYWGQGTLVTVSA

SEQ ID NO: 41 - protein, 686 aa, Homo sapiens
  REGION 1..686: note = Human TG2
AEELVLERCDLELETNGRDHHTADLCREKLVVRRGQPFWLTLHFEGRNYEASVDSLTFSVVTGPAPSQEAGTKARFPLRDAVEEGDWTATVVDQQDCTLSLQLTTPANAPIGLYRLSLEASTGYQGSSFVLGHFILLFNAWCPADAVYLDSEEERQEYVLTQQGFIYQGSAKFIKNIPWNFGQFEDGILDICLILLDVNPKFLKNAGRDCSRRSSPVYVGRVVSGMVNCNDDQGVLLGRWDNNYGDGVSPMSWIGSVDILRRWKNHGCQRVKYGQCWVFAAVACTVLRCLGIPTRVVTNYNSAHDQNSNLLIEYFRNEFGEIQGDKSEMIWNFHCWVESWMTRPDLQPGYEGWQALDPTPQEKSEGTYCCGPVPVRAIKEGDLSTKYDAPFVFAEVNADVVDWIQQDDGSVHKSINRSLIVGLKISTKSVGRDEREDITHTYKYPEGSSEEREAFTRANHLNKLAEKEETGMAMRIRVGQSMNMGSDFDVFAHITNNTAEEYVCRLLLCARTVSYNGILGPECGTKYLLNLNLEPFSEKSVPLCILYEKYRDCLTESNLIKVRALLVEPVINSYLLAERDLYLENPEIKIRILGEPKQKRKLVAEVSLQNPLPVALEGCTFTVEGAGLTEEQKTVEIPDPVEAGEEVKVRMDLLPLHMGLHKLVVNFESDKLKAVKGFRNVIIGPA

SEQ ID NO: 42 - protein, 17 aa, synthetic construct
TISVGGGKTYYPDSVKG